(19) United States
(12) Patent Application Publication
(10) Pub. No.: US 2004/0191168 A1
(43) Pub. Date: Sep. 30, 2004

(54) Tumor cell killing by cell cycle checkpoint abrogation combined with Inhibition of the classical mitogen activated protein (map) kinase pathway

(76) Inventors: Paul Dent, Glen Allen, VA (US); Steven Grant, Richmond, VA (US); Robert McKinstry, Richmond, VA (US); Yun Dai, Richmond, VA (US); Chunrong Yu, Richmond, VA (US)

Correspondence Address: Michael E Whitham, Whitham Curtis & Christofferson, Suite 340, 11491 Sunset Hills Road, Reston, VA, 20190, US

(21) Appl. No.: 10/363,993

(22) PCT Filed: Sep. 28, 2001
(86) PCT No.: PCT/US01/30508

Related U.S. Application Data
(60) Provisional application No. 60/235,938, filed on Sep. 28, 2000.

(51) Int. Cl. A61K 51/00; A61K 31/522

(52) U.S. Cl. 424/1.49; 514/263.31; 514/453

(57) ABSTRACT

The present invention provides a method for treating cancer by promoting apoptosis and reducing clonogenic survival of cancer cells. The method encompasses co-administering 1) a cell cycle checkpoint abrogation agent (for example, UCN-01 or caffeine) and 2) an inhibitor of a compensatory cytoprotective pathway, such as an agent that inhibits the MEK 1/2 pathway (e.g. PD98059, U0126, or PD184352) or an agent that inhibits the PI 3 pathway (e.g. LIY294002 or wortmanin). In addition, because the co-administration step also radiosensitizes cancer cells, the method additionally encompasses the administration of radiation to further reduce clonogenic survival of cancer cells. The method promotes apoptosis and reduces clonogenic survival in many types of cancer cells, including leukemia cells, prostate cancer cells, breast cancer cells, myeloma cells, and lymphoma cells.

### BACKGROUND OF THE INVENTION

1. Field of the Invention

The invention generally relates to the promotion of apoptosis and clonogenic cell death in cancer cells. In particular, the invention provides methods to promote apoptosis in cancer cells by the co-administration of a cell cycle checkpoint abrogation agent and an inhibitor of a compensatory cytoprotective pathway such as the MEK1/2 pathway or the PI 3 pathway. The invention further provides a method of radio-sensitizing cancer cells by the co-administration of a cell cycle checkpoint abrogation agent and an inhibitor of a compensatory cytoprotective pathway such as the MEK1/2 pathway or the PI3 pathway.

2. Background of the Invention

Cancer represents a leading cause of death in The United States. A variety of therapeutic modalities, including surgery, chemotherapy, endocrine ablation, and ionizing radiation have been used in the treatment of various types of cancer with variable success. Unfortunately, cancer cells are often resistant to many commonly employed chemo- and radio-therapeutic strategies. In particular, in contrast to malignant hematopoietic cells, which are programmed to undergo cell death in response to multiple stimuli, the apoptotic machinery is often lacking or defective in epithelial carcinoma cells. In fact, it has been postulated that the relative resistance of epithelial carcinoma cells to apoptosis may account for or contribute to the poor response of such tumors to various therapeutic interventions (Wouters et al. 1999). Thus, further attempts to enhance the susceptibility of epithelial and other types of carcinoma cells to apoptosis are warranted.

UCN-01 (7-hydroxystaurosporine) is a derivative of staurosporine that is currently being evaluated as an anti-neoplastic agent in phase I clinical trials, both alone and in combination with chemotherapeutic agents and ionizing radiation. UCN-01 was originally developed as an inhibitor of PKC 3 (Mizuno et al. 1995). However, UCN-01 has since been shown to inhibit other kinases, including Chk1, which is responsible for phosphorylation, binding to 14-3-3 proteins, and subsequent degradation of the cdc25c phosphatase (Graves et al., 2000). Degradation of cdc25c results in phosphorylation and inactivation of CDKs such as CDK1 (p34 cdc2), which are critically involved in cell cycle arrest after DNA damage and other insults (Peng et al., 1997). In this way, UCN-01 acts as a checkpoint abrogator, an action that may account for its ability to enhance the lethal actions of various cytotoxic agents, including cisplatin (Bunch and Eastman, 1996), mitomycin C (Alcinaga et al, 1993), camptothecin (Shao et al., 1997), fludara-bine (Harvey et al, 2001), gemcitabine (Shi et al., 2001), and 1-b-D-arabinofuranosylcytosine (Tang et al., 2000; Wang et al., 1997), among others. When administered alone, UCN-01 induces arrest in G₂M or G₀G₁, depending upon cell type, or, alternatively, the p53 or pRb status of the cell (Alcinaga et al., 1994; Chen et al., 1999). UCN-01 is also a potent inducer of apoptosis, particularly in hematopoietic cells, a phenomenon that appears to be more closely related to dephosphorylation of CDKs than to inhibition of PKC (Wang et al., 1995).

Phase I and pharmacokinetic studies of UCN-01 have shown that this compound exhibits a very long plasma half-life, presumably a consequence of extensive binding to acidic glycoprotein (Fuse et al., 1999). Nevertheless, free plasma levels of UCN-01 capable of inhibiting Chk 1 and abrogating checkpoint control events appear to be achievable (Kurata et al, 1999; Wilson et al, 2000). In a preliminary study (Wilson et al, 2000), combination of UCN-01 with established cytotoxic agents was associated with evidence of clinical activity in a patient with advanced non-Hodgkin's lymphoma, raising the possibility that UCN-01 may enhance the in vivo activity of conventional chemotherapeutic drugs.

Despite the intense interest in UCN-01 as an antineoplastic agent, the mechanism(s) by which it induces cell death remain(s) incompletely understood. Recently, considerable attention has focused on the role of signal transduction pathways in the regulation of cell survival, particularly those related to three parallel MAPK modules. Of these, the SAPK/JNK and p38 kinase are primarily induced by environmental insults (e.g., DNA damage or osmotic stress) and are generally associated with pro-apoptotic actions (Leppa and Bohmann, 1999; Verheij et al., 1996). In contrast, p42/44 MAPKs (ERKs) are induced by mitogenic or differentiation-related stimuli and are most frequently (although not invariably) associated with pro-survival activity (Segar and Krebs, 1995; Cross et al., 2000). In fact, there is evidence that the relative outputs of the JNK and p42/44 MAPK cascades determine whether a cell lives or dies in response to a noxious stimulus (e.g., growth factor deprivation; Xia et al, 1995). p42/44 MAPK lies downstream of a signaling pathway consisting of PKC, Raf-1, and MEK1 (Tibbles and Woodgett, 1999). Investigation of the functional role of p42/44 MAPK in cell death decisions, as well as other biological processes, has been greatly facilitated by the development of pharmacological MEK inhibitors, including PD98059 (Dudley et al. 1995), U0126 (Favata et al., 1998), and SL327 (Davis et al., 2000). Recently, Seybolt-Leopold et al. (1999) described a novel MEK inhibitor, PD184352, which is able to block MAPK activation and to inhibit the in vivo growth of colon tumor cells in mice. Aside from their intrinsic antitumor activity, MEK inhibitors may also have a role as potentiators of established chemotherapeutic drug action (Jarvis et al., 1998).

Given the fact that UCN-01 can function as a PKC inhibitor (1) and that it has been shown to mimic some of the actions of the PKC down-regulator bryostatin 1 as well as the kinase inhibitor staurosporine (Davis et al., 2000), the possibility that UCN-01 might block the downstream PKC targets MEK1/2 and MAPK appeared plausible. To address this issue, we have examined the apoptotic actions of UCN-01 in relation to its effects on the MEK/MAPK cascade. Contrary to expectations, exposure of cancer cells to submicromolar concentrations of UCN-01 potentiated, rather than reduced, MAPK phosphorylation/activation. Moreover, the combined exposure of cancer cells to UCN-01 and a pharmacological MEK 1/2 inhibitor (such as PD98059, U0126, and PD184352) resulted in a striking, highly synergistic enhancement of apoptosis in the cancer cells. Accompanying phenomena such as mitochondrial damage, caspase activation, and loss of clonogenic survival were also observed. Furthermore, combined treatment with UCN-01 and MEK1/2 inhibitors enhanced the radio-sensitivity of tumor cells in clonogenic survival assays in response to low dose (2 Gy) irradiation.

### SUMMARY OF THE INVENTION

It is an object of this invention to provide a method for promoting apoptosis and reducing clonogenic survival in cancer cells. The method involves the co-administration of a cell cycle checkpoint abrogation agent and an inhibitor of a compensatory cytoprotective pathway. Examples of cell cycle checkpoint abrogation agents include, for example, UCN-01 and caffeine. Examples of inhibitors of compensatory cytoprotective pathways include but are not limited to agents that inhibit the MEK 1/2 pathway (such as PD98059, U0126, and PD 184352) and agents that inhibit the PI 3 pathway. The method successfully promotes apoptosis in many types of cancer cells, including leukemia cells, prostate cancer cells, breast cancer cells, myeloma cells, and lymphoma cells. The method may further comprise the step of exposing the cancer cells to radiation. Irradiation may be via a Cobalt 60 source, implanted radioactive seeds (brachytherapy) or external beam X-rays.

It is a further object of the present invention to provide a method for the treatment of cancer in a patient. The method involves co-administering to the patient a cell cycle checkpoint abrogation agent and an inhibitor of a compensatory cytoprotective pathway. Examples of cell cycle checkpoint abrogation agents that may be used in the practice of the method include, for example, UCN-01 and caffeine. Examples of inhibitors of compensatory cytoprotective pathways that may be used in the practice of the method include but are not limited to agents that inhibit the MEK 1/2 pathway (such as PD98059, U0126, and PD184352) and agents that inhibit the PI 3 pathway such as LY294002 and Wortmanin. Many types of cancer may be treated by the method, including leukemias, prostate cancer, breast cancer, myelomas, and lymphomas. The method may further comprise the step of exposing the cancer cells to radiation. Irradiation may be via a Cobalt 60 source, implanted radioactive seeds (brachytherapy) or external beam X-rays.

It is a further object of the present invention to provide a method of radiosensitizing cancer cells by co-administering to the cancer cells a cell cycle checkpoint abrogation agent and an inhibitor of a compensatory cytoprotective pathway prior to exposing the cells to radiation. Examples of cell cycle checkpoint abrogation agents that may be used in the practice of the method include, for example, UCN-01 and caffeine. Examples of inhibitors of compensatory cytoprotective pathways that may be used in the practice of the method include but are not limited to agents that inhibit the MEK 1/2 pathway (such as PD98059, U0126, and PD184352) and agents that inhibit the PI 3 pathway such as LY294002 and Wortmanin. Irradiation may be via a Cobalt 60 source, implanted radioactive seeds (brachytherapy) or external beam X-rays.

It is a further object of the present invention to provide a composition for use in carrying out the methods of the present invention. The composition comprises a cell cycle checkpoint abrogation agent, an inhibitor of a compensatory cytoprotective pathway, and a carrier suitable for in vivo administration. Examples of cell cycle checkpoint abrogation agents that may be used in the composition include, for example, UCN-01 and caffeine. Examples of inhibitors of compensatory cytoprotective pathways that may be used in the composition include but are not limited to agents that inhibit the MEK 1/2 pathway (such as PD98059, U0126, and PD184352) and agents that inhibit the PI 3 pathway.

### BRIEF DESCRIPTION OF THE DRAWINGS

FIG. 1. A, logarithmically growing U937 cells were incubated for 18 h in the presence of 150 nM UCN-01±10 μM PD184352 cells were treated with 10 μM PD184352 (PD) and/or 150 nM UCN-01 (UCN)±1 mM CHX, after which Wright Giemsa-stained cytospin preparations were evaluated by light microscopy, and the percentage of cells exhibiting classic apoptotic features was determined by examining 5-10 randomly selected fields encompassing ≧500 cells. Values represent the means ±SD for three separate experiments performed in triplicate. B, cells were treated with UCN-01±PD 184352 (±1 mM cycloheximide) as above, after which the percentage of cells exhibiting reduced mitochondrial membrane potential (Δψm) was determined by monitoring DiOC6 uptake as described in “Materials and Methods.” Results represent the means ±SD for three separate experiments performed in triplicate. C, U937 cells were exposed to UCN-01 (200 nM; UCN) 6 PD98059 (50 μM; PD98) for 24 h, after which the percentage of apoptotic cells was scored as described above D, cells were exposed to UCN-01 (200 nM)±20 μM U0126 (UO1) for 24 h, after which apoptosis was determined as above.

FIG. 2. A, HL-60 promyelocytic leukemia cells, Jurkat and CCRF-CEM lymphoblastic leukemia cells, and Raji B-lymphoblastic leukemia cells were exposed to PD184352 (PD;5 μM)±UCN-01 (UCN; 300 nM HL-60; 150 nM Jurkat; 200 nM CCRF; 200 nM Raji) for 24 h, after which the percentage of apoptotic cells was determined as described above. Values represent the means ±SD for three separate experiments performed in triplicate. B and C, U937 cells were exposed to PD184352 (PD;10 μM)+UCN-01 (UCN; 150 nM) for 10 h in the presence or absence of the broad caspase inhibitor ZVAD-fmk (20 μM)or the caspase-8 inhibitor IETD-fmk (20 μM). At the end of this period, cytospin preparations were monitored for apoptosis by morphological examination of Wright Giemsa-stained specimens (A) or the percentage of cells displaying a reduction in Δψm (C) determined by flow cytometry as described in “Materials and Methods.” Values represent the means ±SD for three separate experiments performed in triplicate.

FIG. 3. A, U937 cells were exposed to UCN-01 (UCN; 150 nM)±PD1984352 (D;10 μM) for 12 h and/or 18 h, after which the percentage of cells in G₀G₁,G₂M, S-phase, or the subdiploid fraction (Ap) was determined as described in “Materials and Methods.” B, alternatively, cells were exposed to the same agents for 12 h or 18 h, after which the percentage of BrdUrd FITC-positive (S-phase) cells was determined by flow cytometry as described in “Materials and Methods.” The values represent the means ±SD for three separate experiments performed in triplicate. p, significantly greater than values for control; P≦0.05; **, P≦0.02. C, cells were treated as above for 18 h, after which cdk1/cdc2 activity (expressed as cpm of [γ³²P] incorporated into histone H1) was determined by cdk1/cdc2 kinase assay as described in “Materials and Methods.” Values represent the means ±SD for three separate experiments. p, significantly greater than values for UCN-01 alone; P≦0.05.

FIG. 4. A, cells were treated with PD184352 (PD;10 μM)±caffeine (2 mM) for 18 h, after which the percentage of apoptotic cells and cells exhibiting a reduction in ΔΨm determined by morphological assessment or flow cytometry respectively. Values represent the means ±SD for three separate experiments performed in triplicate. B, U937 cells stably expressing an empty vector (pREP4) and a p21 CIP1 antiserise construct (p21AS) were exposed to UCN-01 (UCN; 150 nM)±PD 184352 (PD; 10 μM) for 18 h, after which the percentage of apoptotic cells was determined by morphological examination as described previously. Values represent the means ±SD for three separate experiments performed in triplicate. p, significantly greater than values for pREP4 cells; P≦0.05; ** P≦0.02. C, cells were exposed to PD184352 and UCN-01 as above for 18 h in the presence or absence of the p38 MAPK inhibitor SB203580 (10 μM), after which the percentage of cells exhibiting the morphological features of apoptosis or reduction in Δψm, reflected by a diminished uptake of DiOC₆, was determined as described previously. Values represent the means ±SD for three separate experiments performed in triplicate. **, significantly less than values for UCN+PD without SB203580; P≦0.02.

FIG. 5. A, cells were exposed to PD184352 (5 μM) UCN-01 (100 nM) for 18 h, after which cells were washed free of drug and plated in soft agar as described in the text. After 12 days of incubation, colonies, consisting of groups of ≧50 cells, were scored, and colony formation for each condition was expressed relative to untreated control cells. Values represent the means ±SD for three separate experiments. B, U937 cells were exposed to a range of PD184352 (e.g., 3.75-10 μM) and UCN-01 (e.g., 75-200 nM) concentrations alone and in combination at fixed ratio (e.g., 50:1) for 18 h. At the end of this period, colony formation was determined for each condition as above. Alternatively, cell viability was determined using the cell titer 96 reagent as described in “Materials and Methods.” In each case, the fraction affected values were determined by comparing results with those of untreated controls, and median dose-effect analysis was used to characterize the nature of the interaction between UCN-01 and PD184352 using a commercially available program (CalcuSyn; Biosoft). ●, values obtained for clonogenic assays; ▾, values obtained for viability assays. Combination index values less than 1.0 denote a synergistic interaction. Two additional studies yielded equivalent results. C, normal peripheral blood mononuclear cells were exposed to 150 nM UCN-01±10 μM PD184352 for 18 h, after which the percentage of apoptotic cells was determined by morphological examination as described previously. Values represent the means ±SD for triplicate determination; a second independent study yielded equivalent results.

FIG. 6. Prolonged activation of MAPK by UCN-01 in mammary and prostate carcinoma cells. Cells were cultured as described in Methods. Panel A. MDA-MB-231 cells. Panel B. MCF7 cells. Panel C. DU145 cells. Panel D. T47D cells. Cells were pre-treated for 30 min with MEK1/2 inhibitor (25 μM) followed at “time 0” by UCN-01 (150 nM) and MAPK activity determined over the next 0-2880 min as in Methods. Cells were lysed and portions (˜100 μg) from each plate used to immunoprecipitate MAPK followed by immune-complex kinase assays measuring increases in ³²P-incorporation into MBP substrate as in Methods. Phosphorylation status did not alter the ability of our antibody to immunoprecipitate MAPK (not shown). MAPK activity data are shown as specific activity (fmol/min/mg), and are from the means ±SEM of 3 independent experiments with MAPK activity values which differed by less than 20%.

FIG. 7. Combined exposure of mammary and prostate carcinoma cells to UCN-01 and MEK1/2 inhibitors causes apoptosis. Cells were either treated with vehicle or with ZVAD (20 μM). In parallel, cells were incubated with matched vehicle control (DMSO), with 25 μM PD98059 alone, with 150 nM UCN-01 alone, or with 25 μM PD98059 and 150 nM UCN-01. Cells were either exposed to radiation (2 Gy) or mock irradiated. Portions of cells were taken 24 hours post irradiation and fixed onto slides by cytocentrifuge followed by staining for double stranded DNA breaks as in Methods. Panel A. MDA-MB-231 cells; Panel B. MCF7 cells; Panel C. DU145 cells; Panel D. T47D cells; Panel E. LNCaP cells; Panel F. Time course of apoptosis for MDA-MB-231 and MCF7 cells treated with 25 μM is PD98059 and 150 nM UCN-01 (cf Panels A and B). Data shown are the mean number of staining cells from randomly selected fields of fixed cells (n=5 per slide, 3 parallel individual experiments ±SEM) which were examined and counted via flourescent light microscopy. #p<0.05 greater than corresponding value in unirradiated cells; *p<0.05 greater than control value.

FIG. 8. The MEK1/2 inhibitors U0126 and PD184352 also increase apoptosis in carcinoma cells treated with UCN-01. Cells were incubated with matched vehicle control (DMSO), with U0126 (5 μM) or PD184352 (10 μM) alone, with 150 nM UCN-01 alone, or with U0126 (5 μM) or PD184352 (10 μM) and 150 nM UCN-01. Cells were either exposed to radiation (2 Gy) or mock irradiated. Portions of cells were taken 24 hours post irradiation and fixed onto slides by cytocentrifuge followed by staining for double stranded DNA breaks as in Methods. Panel A. MDA-MB-231 cells and Panel B. MCF7 cells. Data shown are the mean number of staining cells from randomly selected fields of fixed cells (n=5 per slide, 3 parallel individual experiments ±SEM) which were examined and counted via flourescent light microscopy. *p<0.05 greater than control value.

FIG. 9. Combined treatment of MDA-MB-231 cells with UCN-01 and MEK1/2 inhibitors enhances the cleavage of pro-caspases. Cells were incubated with matched vehicle control (DMSO), with 25 μM PD98059 alone, with 150 nM UCN-01 alone, or with 25 μM PD98059 and 150 nM UCN-01. Figure shows the activity of Cdc2 24 hours after drug treatment. Data shown are the mean activity from 3 parallel individual experiments (±SEM). *p<0.05 greater than control value; #p<0.05 greater than UCN-01 alone value. Identical parallel data were obtained in immune complex assays for the G1/S cyclin dependent kinase, Cdk2 (not shown).

FIG. 10. Combined treatment of MDA-MB-231 cells with UCN-01 and MEK1/2 inhibitors causes a loss of the mitochondrial membrane permeability transition and release of cytochrome c into the cytosol. Cells were either treated with vehicle or with ZVAD (20 μM). In parallel, cells were incubated with matched vehicle control (DMSO), with 25 μM PD98059 alone, with 150 nM UCN-01 alone, or with 25 μM PD98059 and 150 nM UCN-01. Portions of cells were taken 6 and 24 hours post treatment and the mitochondrial membrane potential determined. Data are the means of 3 parallel individual experiments±SEM. *p<0.05 greater than control value.

FIG. 11. The potentiation of apoptosis by combined UCN-01 treatment/MEK1/2 inhibition requires both caspase 9 and caspase 8 functions. Cells were either treated with either vehicle, with IETD (20 μM), with LEHD (20 μM) or with both IETD and LEHD. In parallel, cells were incubated with matched vehicle control (DMSO), with 25 μM PD98059 alone, with 150 nM UCN-01 alone, or with 25 μM PD98059 and 150 μM UCN-01. Cells were either exposed to radiation (2 Gy) or mock irradiated. Portions of cells were taken 24 hours post irradiation and fixed onto slides by cytocentrifuge followed by staining for double stranded DNA breaks as in Methods. Panel A. MDA-MB-231 cells. Panel B. MCF7 cells. Panel C. T47D cells. Panel D. DU145 cells. Data shown are the mean number of staining cells from randomly selected fields of fixed cells (n=5 per slide, 3 parallel individual experiments±SEM) which were examined and counted via flourescent light microscopy. #p<0.05 greater than corresponding value in unirradiated cells; *p<0.05 greater than control value; % p<0.05 less than corresponding value in cells not treated with caspase inhibitor.

FIG. 12. Over-expression of Bcl-_{XL} protects carcinoma cells from the toxic effects of combined UCN-01 and MEK1/2 inhibitor treatment. Cells were infected with recombinant adenoviruses to express either null (CMV), Bcl-2 or Bcl-_{XL}. Twenty four h after infection, cells were incubated with matched vehicle control (DMSO), with 25 μM PD98059 alone, with 150 nM UCN-01 alone, or with 25 μM PD98059 and 150 nM UCN-01. Portions of cells were taken 24 hours post treatment and fixed onto slides by cytocentrifuge followed by staining for double stranded DNA breaks as in Methods. Panel A. MDA-MB-231 cells. Panel B. MCF7 cells. Panel C. DU145 cells. Data shown are the mean number of staining cells from randomly selected fields of fixed cells (n=5 per slide, 3 parallel individual experiments±SEM) which were examined and counted via flourescent light microscopy. #p<0.05 greater than corresponding value in unirradiated cells; *p<0.05 greater than control value.

FIG. 13. Combined treatment of carcinoma cells with UCN-01 and MEK1/2 inhibitors depletes cell numbers in G2/M phase and increases sub-G1 DNA fragmentation. Cells were incubated with matched vehicle control (DMSO), with 25 μM PD98059 alone, with 150 nM UCN-01 alone, or with 25 μM PD98059 and 150 nM UCN-01. Cells were either exposed to radiation (2 Gy) or mock irradiated. Portions of cells were taken 24 hours post irradiation, fixed, digested with RNAase, stained with propidium iodide and flow cytometric analysis performed to assess cell cycle progression of MDA-MB-231 cells. Data shown are the means of duplicate determinations (n=3±SEM). *p<0.05 greater than control value; % p<0.05 less than corresponding value in cells.

FIG. 14. Treatment of U937 human leukemia cells with UCN-01 in combination with the PI3K inhibitor LY294002 results in a marked increase in apoptosis. U937 monocytic leukemia cells were incubated for 24 hr with 100 nM UCN-01 alone, 10 μM LY294002, or the combination, after which the extent of apoptosis was determined by morphological examination. It can be seen that the combination of UCN-01 and LY294002 resulted in a marked increase in the percentage of apoptotic cells.

### ABBREVIATIONS

The abbreviations used are: PKC, protein kinase C; CDK, cyclin-dependent kinase; MAPK, mitogen-activated protein kinase; MEK, MAPK kinase; SAPK, stress-activated protein kinase; JNK, c-Jun NH2 -terminal kinase; ERK, extracellular regulated kinase; TUNEL, terminal deoxynucleotidyl transferase-mediated nick end labeling; DiOC6, 3,3-dihexyloxacarbocynine; BrdUrd, bromodeoxyuridine; CREB, cyclic AMP-responsive element binding protein; PARP, poly(ADP-ribose) polymerase; RIPA, radioimmunoprecipitation assay; CHX, cycloheximide; GFX, bisindolylmaleimide; PMA, phorbol 12-myristate 13-acetate.

### DETAILED DESCRIPTION OF THE PREFERRED EMBODIMENTS OF THE INVENTION

The present invention provides methods for promoting apoptosis and reduced clonogenic survival in cancer cells. The method involves the co-administration of an a cell cycle checkpoint abrogation agent and an inhibitor of a compensatory cytoprotective pathway.

This invention is the result of the unexpected discovery that, contrary to expectations, exposure of cancer cells to submicromolar concentrations of a cell cycle checkpoint abrogation agent potentiate MAPK phosphorylation/activation. Moreover, the combined exposure of cancer cells to a cell cycle checkpoint abrogation agent and a pharmacological inhibitor of a compensatory cytoprotective pathway (such as an agent that inhibits the MEK 1/2 pathway or the PI 3 kinase pathway) resulted in a striling, highly synergistic enhancement of apoptosis in the cancer cells, and this to an extent significantly greater than that which is observed with either agent alone. Furthermore, the co-administration results in mitochondrial damage, caspase activation, and loss of clonogenic survival, events that are typically associated with apoptosis.

The present invention further provides a method of treating cancer in a patient by co-administering a cell cycle checkpoint abrogation agent and an inhibitor of a compensatory cytoprotective pathway to the patient.

By “a cell cycle checkpoint abrogation agent” we mean that the primary activity of the compound, as recognized by those of skill in the art, is to block the normal regulatory growth arrest mechanisms that cells employ to stop growth during times of stress/when their DNA is damaged. Those of skill in the art will recognize that many types of cell cycle checkpoint abrogation agents exist which can be utilized in the practice of the present invention. Examples of such agents include but are not limited to UCN-01, caffeine and the like. Any cell cycle checkpoint abrogation agent may be utilized in the practice of the present invention, so long as the agent exhibits the property of inducing apoptosis in cancer cells when co-administered with an inhibitor of a compensatory cytoprotective pathway.

By “inhibitor of a compensatory cytoprotective pathway” we mean that the primary activity of the compound, as recognized by those of skill in the art, is to block the basal and stimulated activity of a signal transduction pathway(s) that act to protect cells from death. Those of skill in the art will recognize that two broad categories of such inhibitors exist, namely agents that inhibits the Raf/MEK1/2/ERK (MEK 1/2) pathway and agents that inhibit the PI 3 kinase pathway. Examples of agents that inhibits the Raf/MEK1/2/ERK pathway that may be utilized in the practice of the present invention include but are not limited to PD98059, U0126, SL327 and PD184352. Examples of inhibitors of the PI 3 kinase/Akt pathway that may be utilized in the practice of the present invention include but are not limited to LY294002 and wortmanin. Any agent which is an inhibitor of a compensatory cytoprotective pathway may be utilized in the practice of the present invention, so long as the agent exhibits the property of inducing apoptosis in cancer cells when co-administered with a cell cycle checkpoint abrogation agent.

The agents which are utilized in the present invention may be of many types, including typical “small molecule” pharmaceuticals, proteins, antisense oligonucleotides, and the like. Further, they may be synthetically manufactured by chemical synthetic methods, or using molecular biological techniques, or by any method that results in an agent that is suitable for use in the practice of the invention.

By “co-administration” or “co-administering” we mean that the two agents are administered in temporal juxtaposition. The co-administration may be effected by the two agents being mixed into a single formulation, or by the two agents being administered separately but simultaneously, or separately and within a short time of each other. For example, in general the two agents are co-administered within the time range of 24-72 hours. In this case, the agents may be administered in either order, i.e. the cell cycle checkpoint abrogation agent may be administered first, or the inhibitor of a compensatory cytoprotective pathway may be administered first. In a preferred embodiment of the instant invention, the two agents are co-administered in a single formulation, or are co-administered simultaneously. Further, more than one cell cycle checkpoint abrogation agent or more than one inhibitor of a compensatory cytoprotective pathway may be administered together, and inhibitors of different compensatory cytoprotective pathways may be co-administered together.

By “promoting apoptosis” and “reducing clonogenic survival” we mean that the level of apoptosis and non-apoptotic cell death occurring in the targeted cancer cells upon exposure of the cancer cells to a cell cycle checkpoint abrogation agent and an inhibitor of a compensatory cytoprotective pathway (with or without radiation exposure) is greater than the levels of apoptosis and reduction in clonogenic survival that would occur in the presence of either agent alone. Further, the effect is greater than the mere additive effect of the two agents together would be expected to be from observations of their independent activities i.e. the two agents act synergistically. In general, the increase in the level of apoptosis will be in the range of about 10% to 100%. In a preferred embodiment of the present invention, the increase in the level of apoptosis will be in the range of about 40% to 80%. In yet another preferred embodiment, the increase is in the range of about 70% to 80%. Those of skill in the art will recognize that it is possible to quantitate the level of apoptosis in cancer cells by several means which are well-known and readily available, including morphological assessment of Wright and Giemsa-stained cytospin preparations, TUTNEL, and colony formation assays. The effects may be assessed in vivo or in vitro. In general, the reduction in clonogenic survival of cancer cells will be in the range of about 30-70%. In a preferred embodiment, the reduction in clonogenic survival of cancer cells will be in the range of about 60 to 70%.

While radiation does not appear to significantly increase the apoptotic response of tumor cells beyond that of the drug exposure alone (Example 11), it does synergistically interact to markedly reduce the ability of surviving cells to proliferate when combined with the drugs (Example 16). Thus, in several aspects of the instant invention, the co-administration of the two subject agents is coupled with the further step of administering radiation.. In one embodiment of the instant invention, radiation is administered after co-administration of the two agents. In general, the radiation is administered from 0 to 24 hours after treatment with the agents. Those of skill in the art will recognize that many means exist for the administration of radiation, including but not limited to a single beam, implanted seed single dose (brachytherapy), multiple fractionated external beam doses, etc. Protocols for the administration of radiation are well known and readily available to those of skill in the art. These include established protocols for the administration of drugs in combination with radiation therapy (Wobst et al. 1998). Further, as those of skill in the art will recognize, the details of coupling the administration of radiation with the co-administration of a cell cycle checkpoint abrogation agent and an inhibitor of a compensatory cytoprotective pathway is normally refined under the direct supervision of a physician during clinical trials.

The present invention also provides a method of radiosensitizing cancer cells. By “radiosensitizing cancer cells” we mean that a desired effect of treating cancer cells with radiation (e.g. reducing the clonogenic survival of the cancer cells) is promoted or augmented, such that the effect is more marked than when the cancer cells are treated with radiation alone. The method of radiosensitizing cancer cells in the present invention involves co-administering a cell cycle checkpoint abrogation agent and an inhibitor of a compensatory cytoprotective pathway prior to administering the radiation. The nature of the two co-administered agents and the radiation is that which is described herein for the methods of promoting apoptosis and reduced clonogenic survival in cancer cells and for treating cancer.

The methods described herein can be used for promoting apoptosis in and treating cancers of a number of types, including but not limited to breast and prostate cancer, brain cancer, sarcomas and other neoplasms, bladder cancer, colon cancer, lung cancer, various leukemias and lymphomas, multiple myeloma etc. Further, the methods of the present invention may be used to treat cancer in humans, and may also be utilized in the treatment of other species, i.e. may also be used for veterinary purposes.

One skilled in the art will recognize that the amount of a cell cycle checkpoint abrogation agent and an inhibitor of a compensatory cytoprotective pathway to be co-administered will be that amount sufficient to promote apoptosis in the targeted cancer cells. Such an amount may vary inter alia depending on such factors as the gender, age, weight, overall physical condition, of the patient, etc. and must be determined on a case by case basis. The amount may also vary according to the type of cancer being treated, and the other components of the treatment protocol (e.g. other forms of chemotherapy, surgery, and the like. It is expected that serum concentrations (or localized concentrations at the site of a tumor) of either agent in the range of about 10 nM to 500 nM would be sufficient in most cases. In some embodiments of the instant invention, a concentration range of about 50 nM to about 250 nM is preferable. In a preferred embodiment of the present invention, the concentration of agent is about 100 to 200 nM. Those of skill in the art will recognize that such details are normally worked out during clinical trials.

Co-administration of the agents may be oral, perenteral or topical. The term parenteral as used herein includes subcutaneous injections, intravenous, intramuscular, intraarterial injection, or infusion techniques. The agents may be administered in any of several forms, including tablets, pills, powders, lozenges, sachets, cachets, elixirs, suspensions, emulsions, solutions, syrups, aerosol (as a solid or in a liquid medium), soft or hard gelatin capsules, suppositories, sterile injectable solutions and sterile packaged powders.

All pharmaceutical compositions of the agents utilized in the practice of the present invention may also include a pharmaceutically acceptable carrier. The agents may be mixed with a carrier, or diluted by a carrier, or enclosed within a carrier which may be in the form of a capsule, sachet, paper or other container. When the carrier is a diluent, it may be a solid, semisolid or liquid material which acts as a vehicle, excipient or medium for the inhibitor. Some examples of suitable carriers, excipients and diluents include lactose, dextrose, sucrose, sorbitol, mannitol, starches, gum acacia, calcium phosphates, alginate, tragacanth, gelatin, calcium silicate, microcrystalline cellulose, polyvinylpyrrolidone, celluose, water syrup, methyl cellulose, methyl and propylhydroxybenzoates, talc, magnesium stearate and mineral oil. The formulations can also include lubricating agents, wetting agents, emulsifying agents, preservatives, and sweetening or flavoring agents.

Injectable preparations, for example, sterile injectable aqueous or oleaginous suspensions may be formulated according to the known art using suitable dispensing or wetting agents and suspending agents. The sterile injectable preparation can also be a sterile injectable solution or suspension in a nontoxic parenterally acceptable diluent or solvent, for example, as a solution in 1,3-butanediol.

In another aspect, the present invention contemplates a pharmaceutical composition comprising a cell cycle checkpoint abrogation agent, an inhibitor of a compensatory cytoprotective pathway, and a carrier suitable for in vivo administration of the composition. Examples of cell cycle checkpoint abrogation agents include but are not limited to UCN-01, caffeine etc.. Examples of inhibitors of compensatory cytoprotective pathways include but are not limited to agents that inhibit the MEK 1/2 pathway such as PD98059, U0126, PD184352, SL327, and agents that inhibit the PI3 pathway. Such a composition also comprises a carrier suitable for in vivo administration, examples of which are listed above.

The following examples provide illustrations of the practice of the present invention but should not be construed so as to limit the invention in any way.

### EXAMPLES

### Materials and Methods for Examples 1-9

Cells. U937, BL-60, Jurkat, CCRF-CEM, and Raji cells are human histiocytic lymphoma, acute promyelocytic leukemia, acute T-cell leukemia, acute lymphoblastic leukemia, and Burkitt lymphoma cell lines, respectively. All of the cells were derived by the American Type Culture Collection and maintained in RPMI 1640 medium containing 10% FBS, 200 units/ml penicillin, 200 mg/ml streptomycin, minimal essential vitanins, sodium pyruvate, and glutaine, as reported previously (Vrana and Grant, 2001). U937/p21AS and U937/pREP4 cells were obtained by stable transfection of cells with plasmids containing anti-sense-oriented p21 cDNA or an empty vector (pREP4), and clones were selected with hygromycin (Wang et al, 1999).

Drugs and Reagents. Selective MEK inhibitors (PD98059 and U0126), selective PKC inhibitors (GF 109203X or GFX I and safingol), and specific inhibitors of p38 MAPK (SB203580) were supplied by Calbiochem (San Diego, Calif.) as powder. The MEK inhibitor PD184352 was from Warner Lambert/Parke-Davis Co., Ann Arbor, Mich. Materials were dissolved in sterile DMSO and stored frozen under light-protected conditions at −20° C. UCN-01 was kindly provided by the Developmental Therapeutics Program/Cancer Treatment and Evaluation Program (CTEP), National Cancer Institute. It was dissolved in DMSO at a stock concentration of 1 mM, stored at −20° C., and subsequently diluted with serum-free RPMI medium before use. Caffeine (Alexis Co., San Diego, Calif.) was dissolved in chloroform and stored at −20° C. In all of the experiments, the final concentration of DMSO or chloroform did not exceed 0.1%. Caspase inhibitor (Z-VAD-fmlc) and caspase 8 inhibitor (Z-IETD-fmk) were purchased from Enzyme System Products (Livermore, Calif.), dissolved in DMSO, and stored at 4° C. Cycloheximide was purchased from Sigma Chemical Co. (St. Louis, Mo.), stored frozen in DMSO, and diluted in RPMI 1640 medium before use.

Experimental Format. All of the experiments were performed using logarithmically growing cells (3-5×10⁵ cells/ml). Cell suspensions were placed in sterile 25 cm² T-flasks (Corning, Corning, N.Y.) and incubated with MEK or PKC inhibitors for 30 min at 37° C. At the end of this period, UCN-01 (or in some cases, caffeine) was added to the suspension, and the flasks were placed in 37° C./5% CO₂ incubator at various intervals, generally 18 h. In some studies, the p38 MAP kinase inhibitor SB203580 was added concurrently with MEK inhibitors. After drug treatment, cells were harvested and subjected to further analysis as described below.

Analysis of Apoptosis. The extent of apoptosis was evaluated by assessment of Wright-Giemsa-stained preparation under light microscopy and scoring the number of cells exhibiting classic morphological features of apoptosis. For each condition, 5 to 10 randomly selected fields/condition were evaluated, encompassing at least 500 cells (Vrana and Grant, 2001). To confirm the results of morphological analysis, in some cases cells were also evaluated by TUNEL staining (Gorczyca et al., 1993) and assessment of oligonucleosomal DNA fragmentation of total DNA. DNA fragmentation was analyzed by 1.8% agarose gel electrophoresis as described previously (Jarvis et al., 1994). For TUNEL staining, cytocentrifuge preparations were obtained and fixed with 4% formaldehyde. The slides were treated with acetic acid/ethanol (1:2), stained with terminal transferase reaction mixture containing terminal transferase reaction buffer, 0.25 units/ ml terminal transferase, 2.5 mM CoCl₂, and 2 pmol fluorescein-12-dUTP (Boehringer Mannheim, Indianapolis, Ind.), and visualized using fluorescence microscopy.

Analysis of Mitochondrial Membrane Potential (ΔΨm). Cells 2×10⁵ were incubated with 40 nM DiOC₆ (Molecular Probes Inc., Eugene, Oreg.) in PBS at 37° C. for 20 min and then analyzed by flow cytometry as described previously (Wang et al., 1999). The percentage of cells exhibiting a low level of DiOC₆ uptake, which reflects loss of mitochondrial membrane potential, was determined using a Becton Dickinson FACScan (Becton Dickinson, San Jose, Calif.).

Cell Cycle Analysis and S-phase Content. Cells (2×10⁶) were pelleted at 4° C., resuspended, fixed at 4° C. with 67% ethanol overnight, and treated on ice with a propidium iodide solution containing 3.8 mM Na citrate, 0.5 mg/ml RNase A (Sigma Chemical Co.), and 0.01 mg/ml propidium iodide (Sigma Chemical Co.) for 3 h. Cell cycle analysis was performed by flow cytometry using Verity Winlist software (Topsham, Me.). Incorporation of BrdUrd was monitored to evaluate S-phase content. For each condition, 2 3 10 6 cells (cell density 5 5 3 10 5 /ml) were incubated with 10 mM BrdUrd for 30 min at 37° C. After washing twice with 1% BSA/PBS, the cells were resuspended in 70% ethanol and fixed for 30 min on ice. The BrdUrd-labeled cells were denatured and nuclei released by incubation with 2 N HCl/0.5% Triton X-100 for 30 min at room temperature. After centrifugation, the pellet was resuspended in 0.1 M Na₂ B₄O₄ (pH 8.5) to neutralize the acid. Cells (1 3 10 6 )/100 ml in 0.5% Tween 20/1% BSA/PBS were incubated with FITC-conjugated anti-BrdUrd (1:10; mouse monoclonal; DAMO, Carpin-teria, Calif.) for 30 min at 4° C. After washing once with 0.5% Tween 20/1% BSA/PBS, the cells were resuspended in PBS containing 5 mg/ml propiditun iodide and analyzed by flow cytometry. The percentage of S-phase cells was determined by measuring BrdUrd FITC-positive part in a dot plot of FL-3 (red fluorescence) against FL-1 (green fluorescence).

Immunoblot and Immunoprecipitation Analysis. Whole-cell pellets were lysed by sonication in 1× sample buffer [62.5 mM Tris base (pH6.8), 2% SDS, 50 mM DTT, 10% glycerol, 0.1% bromphenol blue, and 5 mg/ml each chymostatin, leupeptin, aprotinin, pepstatin, and soybean trypsin inhibitor] and boiled for 5 min. For analysis of phospho-proteins, 1 mM each Na vanadate and Na PPi was added to the sample buffer. Protein samples were collected from the supernatant after centrifugation of the samples at 12,800 3 g for 5 min, and protein was quantified using Coomassie Protein Assay Reagent (Pierce, Rock-ford, Ill.). Equal amounts of protein (30 mg) were separated by SDS-PAGE and electrotransferred onto a nitrocellulose membrane. For blotting phospho-proteins, no SDS was included in the transfer buffer. The blots were blocked with 5% milk in PBS-Tween 20 (0.1%) at room temperature for 1 h and probed with the appropriate dilution of primary antibody overnight at 4° C. The blots were washed twice in PBS-Tween 20 for 15 min and then incubated with a 1:2000 dilution of horseradish peroxidase-conjugated secondary antibody (Kirkegaard & Perry, Gaithersburg, Md.) in 5% milk/PBS-Tween 20 at room temperature for 1 h. After washing twice in PBS-Tween 20 for 15 min, the proteins were visualized by Western Blot Chemiluminescence Reagent (NEN Life Science Products, Boston, Mass.). For analysis of phospho-proteins, Tris-buffered saline was used instead of PBS throughout. Where indicated, the blots were reprobed with antibodies against actin (Signal Transduction Laboratories) or tubulin (Calbiochem) to ensure equal loading and transfer of proteins. The following antibodies were used as primary antibodies: phospho-p44/42 MAPK (Thr202/Tyr204) antibody (1:1000; rabbit polyclonal; NEB, Beverly, Mass.); p44/42 MAPK antibody (1:1000; rabbit polyclonal; NEB); phospho-p38 MAPK (Thr180/Tyr182) antibody (1:1000; rabbit polyclonal; NEB); phospho-SAPK/JNK (Thr183/Tyr185) antibody (1:1000; rabbit polyclonal; Cell Signaling Technology, Beverly, Mass.); SAPK/JNK antibody (1:1000; rabbit poly-clonal; Cell Signaling Technology); anti-phospho-CREB (1:1000; rabbit polyclonal; Upstate Biotechnology, Lake Placid, N.Y.); phospho-cdc2 (Tyr15) antibody (1:1000; rabbit polyclonal; Cell Signaling Technology); anti-p21Cip/WAF1 (1:500; mouse monoclonal; Transduction Laboratories, Lexington, Ky.); anti-p27kip1 (1:500; mouse monoclonal; PharMingen, San Diego, Calif.); MAP kinase phosphatase-1 (M-18; 1:200; rabbit polyclonal; Santa Cruz Bio-technology Inc., Santa Cruz, Calif.); MAP Kinase phosphatase-3 (C-20; 1:100; goat polyclonal; Santa Cruz Biotechnology Inc.); antihuman Bcl-2 oncoprotein (1:2000; mouse monoclonal; DAKO, Carpinteria, Calif.); Bax (N-20; 1:2000; rabbit polyclonal; Santa Cruz Biotechnology Inc.); Bcl-xS/L (S-18; 1:500; rabbit polyclonal; Santa Cruz Biotechnology Inc.); antihuman/mouse XIAP (1:500; rabbit polyclonal; R&D System, Minneapolis, Minn.); anti-caspase-3 (1:1000; rabbit polyclonal; PharMingen); cleaved-caspase-3 (Mr 17,000) antibody (1:1000; rabbit polyclonal; Cell Signaling Technology); anti-caspase-9 (1:1000; rabbit polyclonal; PharMingen); anti-PARP (1:2500; mouse mono-clonal; Calbiochem); and cleaved PARP (M r 89,000) antibody (1:1000; rabbit polyclonal; Cell Signaling Technology). Immunoprecipitation was performed to determine the extent of cdc25C activation (Peng et al, 1998). Briefly, 2×10⁷cells were lysed in RIPA buffer (1% NP40, 0.5% Na deoxycholate, 1 mM phenylmethylsulfonylfluoride, 1 mM Na vanadate, 5 mg/ml chymostatin, leupeptin, aprotinin, pepstatin, and soybean trypsin inhibitor, and 0.1% SDS in PBS) by syringing approximately 20 times with a 23-gauge needle. Protein samples were centrifuged at 12,800×g for 30 min and quantified. Two-hundred mg of protein/condition were incubated under continuous shaling with 1 mg of anti-cdc25C (mouse monoclonal; PharMin-gen) overnight at 4° C. Twenty ml/condition of Dynabeads (goat antimouse IgG; Dynal, Oslo, Norway) were added and incubated for an additional 4 h. After washing three times with RIPA buffer, the bead-bound protein was eluted by vortexing and boiling in 20 ml of 1× sample buffer. The samples were separated by 12% SDS-PAGE and subjected to immunoblot analysis as described above. Anti-14-3-3 , (rabbit polyclonal; Santa Cruz Biotechnology Inc.) was used as primary antibody at a dilution of 1:200.

Analysis of Cytosolic Cytochrome c. Cells (2×10⁶) were washed in PBS and lysed by incubating for 30 s in lysis buffer (75 mM NaCl, 8 mM Na, HPO₄, 1 mM NaH₂ PO₄, 1 mM EDTA, and 350 mg/ml digitonin). The lysates were centrifuged at 12,000×g for 1 min, and the supernatant was collected and added to an equal volume of 2× sample buffer. The protein samples were quantified, separated by 15% SDS-PAGE, and subjected to immunoblot analysis as described above. Anticytochrome c (mouse monoclonal; PharMingen) was used as primary antibody at a dilution of 1:500.

Cdk1/cdc2 Kinase Assay. Cdk1/cdc2 Kinase Assay Kit Upstate Biotechnology) was used to determine the activity of cdk1/cdc2 kinase according to the manufacturer's instructions. Briefly, 2×10⁷ cells were lysed in RIPA buffer by sonication. Protein samples were centrifuged at 12,800×g for 30 min and quantified. Fifty mg of protein/condition were incubated with 400 mg/ml histone H1, 2 mCi of [γ³²P] ATP, and 1:5 inhibitor cocktail in assay dilution buffer (total volume, 50 ml) at 30° C. for 20 min. A 25- ml aliquot of reaction mixture was transferred onto P81 paper. After washing three times with 0.75% phosphoric acid and once with acetone, cpm of [γ³²P] incorporated into histone H1 was monitored using TRI-CARB 2100TR Liquid Scintillation Analyzer (Packard Instrument Co., Downers Grove, Ill.). In some cases, 10 ml of 2× sample buffer was added to 10 ml of the reaction mixture and boiled for 5 min. [γ³²P] histone H1 was separated by 12% SDS-PAGE and visualized by exposure of the dried gels to X-ray film (KODAK) at −80° C. for 1 h.

Clonogenic Assay and Cell Proliferation Assays. Colony formation after drug treatment was evaluated using a soft agar cloning assay as described previously (Blasina et al, 1999). Briefly, cells were washed three times with serum-free RPMI medium. Subsequently, 500 cells/well were mixed with RPMI medium con-taining 20% FBS and 0.3% agar and plated on 12-well plates (three wells/ condition). The plates were then transferred to a 37° C./5% CO₂, fully humid-ified incubator. After 10 days of incubation, colonies, consisting of groups 5 of 50 cells, were scored using an Olympus Model CK inverted microscope, and colony formation for each condition was calculated in relation to values obtained for untreated control cells. For cell viability assays, CellTiter 96 AQueous One Solution (Promega, Madison, Wis.) was used according to the manufacturer's instructions, and the absorbance at 490 nm was recorded using a 96-well plate reader (Molecular Devices, Sunnyvale, Calif.).

Normal Peripheral Blood Mononuclear Cells. Peripheral blood was obtained with informed consent from normal volunteers, diluted 1:3 with RPMI 1640 medium, and layered over a cushion of 10 ml of Ficoll-Hypaque (specific gravity, 1.077; Sigma Chemical Co.) in sterile 50-ml plastic centrifuge tubes. These studies have been approved by the Human Investigations Committee of Virginia Commonwealth University. After centrifugation for 40 min at 400×g at room temperature, the interface layer, consisting of mononuclear cells, was extracted with a sterile Pasteur pipette and diluted in fresh RPMI medium. The cells were washed twice in medium and resuspended in RPMI 1640 medium containing 10% FCS in 25-cm² tissue culture flasks at a cell density of 10⁶ cells/ml. Various concentrations of UCN-01±PD 184352 were added to the flasks, after which they were placed in the incubator for 24 h. At the end of this period, cytospin preparations were obtained and stained with Wright-Giemsa, and the cells were scored under light microscopy for the typical morphological features of apoptosis.

Statistical Analysis. For morphological assessment of apoptotic cells, cell cycle analysis, S-phase content, cdk1/cdc2 linase assay, analysis of Δψm, and clonogenic and cell proliferation assays, experiments were repeated at least three times. Values represent the means 6 SD for at least three separate experiments performed in triplicate. The significance of differences between experimental variables was determined using the Student t test.

### Materials and Methods for Examples 10-16

Materials. Agarose conjugated anti-p42 MAPK antibody (sc-154-AC) was from Santa Cruz Biotechnology (Santa Cruz Biotechnologies, Calif.). Phospho-p44/42 MAP kinase (Thr202/Tyr204) antibody (1:1000, rabbit polyclonal, NEB, Beverly, Mass.), p44/42 MAP kinase antibody (1:1000, rabbit polyclonal, NEB), phospho-p38 MAP kinase (Thr180/Tyr182) antibody (1:1000, rabbit polyclonal, NEB), anti-phospho-CREB (1:1000, rabbit polyclonal, Upstate Biotechnology, Lake Placid, N.Y.), phospho-cdc2 (Tyr15) antibody (1:1000, rabbit polyclonal, Cell Signaling Technology), anti-p21Cip/WAF1/mda6 (1:500, mouse monoclonal, Transduction Laboratories, Lexington, Ky.), anti-p27kip1 (1:500, mouse monoclonal, Pharmingen, San Diego, Calif.), anti-human Bcl-2 oncoprotein (1:2000, mouse monoclonal, Dalco, Carpinteria, Calif.), Bax N-20, 1:2000, rabbit polyclonal, Santa Cruz Biotechnology Inc.), Bcl-XS/L (S-18, 1:500, rabbit polyclonal, Santa Cruz Biotechnology Inc.), anti-human/mouse XIAP (1:500, rabbit polyclonal, R&D System, Minneapolis, Minn.), anti-cytochrome c (1:500, mouse monoclonal, Pharmingen), anti-caspase-3 (1:1000, rabbit polyclonal, Pharmingen), cleaved-caspase-3 (17 kda) antibody (1:1000, rabbit polyclonal, Cell Signaling Technology), anti-caspase-9 (1:1000, rabbit polyclonal, Pharmingen), anti-PARP (1:2500, mouse monoclonal, Calbiochem), and cleaved PARP (89 kDa) antibody (1:1000, rabbit polyclonal, Cell Signaling Technology). Radiolabelled [γ³²P]-ATP was from NEN. Selective MEK1/2 inhibitors (PD184352, PD98059, and U0126) were supplied by Calbiochem (San Diego, Calif.) as powder, dissolved in sterile DMSO, and stored frozen under light-protected conditions at −20° C. UCN-01 was kindly provided by Dr. Edward Sausville, Developmental Therapeutics Program/CTEP, NCI. It was dissolved in DMSO at a stock concentration of 1 mM, and stored at −20° C., and subsequently diluted with serum-free medium prior to use. Caffeine (Alexis Cor., San Diego, Calif.) was dissolved in chloroform and stored at −20° C. In all experiments, the final concentration of DMSO or chloroform did not exceed 0.1%. Caspase inhibitor (Z-VAD-FMK) and caspase 8 inhibitor (Z-IETD-FMK) were purchased from Enzyme System Products(Livermore, Calif.), dissolved in DMSO and stored at 4° C. Western immunoblotting was performed using the Amersham Enhanced Chemi-Luminescence (E.C.L.) system (Bucks, England). Other reagents were as in [Park et al., 1999].

Generation of primary human mammary epithelial cells. Primary human mammary epithelial cells were isolated from reduction mammoplasty and prepared as described in [Gao, 2001].

Culture of primary mammary cells, and carcinoma cells. Asynchronous carcinoma cells MCF7 (p53+,RB+,ER+); MDA-MB-231 (p53−,RB+,ER−); T47D (p53−,RB+,ER+); DU145 (p53−,RB−,AR−); LNCaP (p53+,RB+,AR+) were cultured in Dulbecco's Modified Eagle's Medium (DMEM) supplemented with 5% (v/v) fetal calf serum at 37° C. in 95% (v/v) air, 5% (v/v) CO₂. Cells were plated at a density 3.2×10⁴ cells/cm² plate area and grown for 36 h prior to further experimentation.

Recombinant adenoviral vectors; generation and infection in vitro. We generated recombinant adenoviruses to express either Bcl-2, Bcl-xl or p21 antisense mRNA. To assess the effectiveness of recombinant adenoviral infection, we generated a recombinant virus containing the gene for β-galactosidase. Cells were infected with this virus after isolation in vitro (m.o.i. 50), and incubated at 37° C. for a further 24 h; cells were fixed and incubated with X-Gal [Valerie et al., 2001]. All cells, infected at an m.o.i. of 50 gave >80% staining.

Exposure of cells to ionizing radiation and cell homogenization. Cells were cultured in DMEM+5% (v/v) fetal calf serum as above. U0126/PD98059/PD184352 treatment was from a 100 mM stock solution and the maximal concentration of vehicle (DMSO) in media was 0.02% (v/v). Cells were irradiated to a total of 2 Gy using a ⁶⁰Co source at dose rate of 2.1 Gy/min. Cells were maintained at 37° C. throughout the experiment except during the ˜1 min irradiation itself. Zero time is designated as the time point at which exposure to radiation ceased. After radiation-treatment cells were incubated for specified times followed by aspiration of media and snap freezing at −70° C. on dry ice. Cells were homogenized in 1 ml ice cold buffer A [25 mM β-glycerophosphate, pH 7.4 at 4° C., 5 mM EDTA, 5 mM EGTA, 5 mM benzamidine, 1 mM phenylmethyl sulphonylfluoride, 1 mg/ml soybean trypsin inhibitor, 40 μg/ml pepstatin A, 1 μM Microcystin-LR, 0.5 mM sodium oldiovanadate, 0.5 mM sodium pyrophosphate, 0.05% (w/v) sodium deoxycholate, 1% (v/v) Triton X100, 0.1% (v/v) 2-mercaptoethanol], with trituration using a P1000 pipette to lyse the cells. Homogenates were stored on ice prior to clarification by centrifugation (4° C.).

Immunoprecipitations from Lysates. Fifty microliters of Protein A agarose (Ag) slurry (25 μl bead volume) was washed twice with 1 ml PBS containing 0.1% (v/v) Tween 20, and resuspended in 0.1 ml of the same buffer. Antibodies (2 μg, 20 μl), serum (20 μl) were added to each tube and incubated (3 h, 4° C.). For pre-conjugated antibodies,10 μl of slurry (4 μg antibody) was used. Clarified equal aliquots of lysates (0.25 ml, 100 μg total protein) were mixed with Ag-conjugated antibodies in duplicate using gentle agitation (2.5 h, 4° C.). Ag-antibody-antigen complexes were recovered by centrifugation, the supernatant discarded, and washed (10 min) sequentially with 0.5 ml buffer A (twice), PBS and buffer B [25 mM Hepes, pH 7.4, 0.1 mM Na₃VO₄].

Assay of p42^{MAPK} activity. Immunoprecipitates were incubated (final volume 50 μl ) with 50 μl of buffer B containing 0.2 mM [γ-³²P]ATP (5000 cpm/pmol), 1 μM Microcystin-LR, 0.5 mg/ml myelin basic protein NP), which initiated reactions at time=0. After 20 min., 40 μl of the reaction mixtures were spotted onto a 2 cm circle of P81 paper (Whatman, Maidstone, England) and immediately placed into 180 mM phosphoric acid. Papers were washed four times (10 min each) with phosphoric acid, and once with acetone, and ³²P-incorporation into MBP was quantified by liquid scintillation spectroscopy.

SDS poly-acrylamide gel electrophoresis (SDS PAGE) and Western blotting. Cells were irradiated and at specified time points/treatments media aspirated and the plates snap frozen. Cells were lysed with homogenization buffer and subjected to immunoprecipitation. Immunoprecipitates were solubilized with 100 μl 5×SDS PAGE sample buffer (10% (w/v) SDS), diluted to 250 μl with distilled water, and placed in a 100° C. dry bath for 15 min. One hundred microliter aliquots of each time point were subjected to SDS PAGE on 10% (w/v) acrylamide gels. Gels were transferred to nitrocellulose by the Method of Towbin and Western blotting using specific antibodies performed as indicated. Blots were developed using Enhanced Chemi-Luminescence (Amersham) using Fuji RX x-ray film. Blots were digitally scanned using Adobe Photoshop, their color removed, and Figures created in Microsoft PowerPoint.

Terminal Uridyl-Nucleotide End Labeling (TUNEL) for apoptosis. Cells were grown in 100 mm dishes as described, treated with or without varying concentrations of U0126/PD184352/PD98059/DMSO control 30 min prior to irradiation and irradiated (2 Gy). Cells were isolated 24 h after irradiation by trypsinizationi followed by centrifugation onto glass slides (cytospin). Terminal Uridyl-Nucleotide End Labeling (TUNEL) was performed on these cells as described previously [Wang et al., 1999; Park et al., 1999]. Randomly selected fields of fixed cells (150 cells per field, n=5 per slide) were counted initially using propidium iodide counter stain, followed by examination and counting of TUNEL positive staining cells of the same field under FITC/fluorescence light.

Cell cycle analysis: propidium iodide staining of cells. Cells were isolated by tryptic digestion at the indicated times after various treatments and aliquots containing 1×10⁶ cells were pelleted by centrifugation at 1500 rpm, 4° C. for 5 min. and resuspended in 1.5 ml of PBS followed by the addition of 3 ml of 100% (v/v) ETOH (67% (v/v) ETOH Final) and incubated on ice at 4° C. for 3 h. Cells were pelleted by centrifugation as above, supernatant removed and resuspended in 1.0 ml of propidium iodide stain containing 3.8 mM sodium citrate, 0.5 mg/ml RNAse A and 0.01 mg/ml propidium iodide and incubated on ice at 4° C. overnight. Cells were pelleted by centrifugation as above, supernatant removed and resuspended in 1.0 ml of PBS. Cells were analyzed with a Becton-Dickinson FACScan flow cytometer and Verity Winlist software.

MTT assay for cell growth. Cells were grown in 12 well plates and 36 h after plating are pre-treated for 30 min with varying concentrations of MEK1/2 inhibitor/DMSO control before further drug treatment/irradiation. Cells were cultured for a further 48 h. A 5 mg/ml stock solution of MTT reagent (3-[4,5-Dimethylthiazol-2-yl]-2,5-diphenyltetrazolium bromide; thiazolyl blue) was prepared in DMEM. For assay of mitochondrial dehydrogenase function, the MTT stock solution is diluted 1:10 in fresh media (DMEM+10% fetal calf serum) and 1 ml of this solution is added to each aspirated well of a 12 well plate. Cells are incubated for a further 3 h at 37° C. MTT is converted into an insoluble purple formazan by cleavage of the tetrazolium ring by mitochondrial dehydrogenase enzymes. After 3 h, media is aspirated and cells lysed with 1 ml DMSO, releasing the purple product from the cells. Cells are incubated for a further 10 min at 37° C. with gentle shaking. Absorbance readings at 540 nM are determined using a computer controlled micro-plate analyzer. The relationship between cell number and MTT absorbance/mitochondrial enzyme activity was linear over the range of 500-10,000 cells.

Analysis of Cytosolic Cytochrome C. 2×10⁶ cells were washed in PBS and lysed by incubating for 30 seconds in lysis buffer (75mM NaCl, 8 mM Na₂HPO₄, 1 mM NaH₂PO₄, 1 mM EDTA, and 350 ug/ml digitonin). The lysates were centrifuged at 12,000 g for 1 min, and supernatant was collected and added to equal volume of 2× sample buffer. The protein samples were quantified, separated by 15% SDS-PAGE, and subjected to immunoblot analysis as described above. Anti-cytochrome c (mouse monoclonal, Pharmingen) was used as primary antibody at a dilution of 1:500.

Analysis of Mitochondrial Membrane Potential (ΔΨm). 2×10⁵ cells were incubated with 40 nM 3,3-dihexyloxacarbocynine (DiOC6, Molecular Probes Inc. Eugene, Oreg.) in PBS at 37° C. for 20 min, and then analyzed by flow cytometry as described preciously [24]. The percentage of cells exhibiting low level of DiOC6 up-take, which reflects loss of mitochondrial membrane potential, was determined using a Becton-Dickinson FACScan analyzer.

Colony forming (clonogenic) assay. Cells were plated 36 h prior to experimentation. Cells were pre-treated with MEK1/2 inhibitor as indicated, 2 h prior to exposure. Cells were irradiated (2 Gy). After a further 48 h, cells were isolated by tryptic digestion and single cell suspensions plated on Linbro® plates at densities of 500 cells/well and 1000 cells/well. Colony formation was defined as a colony of 50 cells or greater, 10 days after plating.

Data analysis. Comparison of the effects of treatnents was done using one way analysis of variance and a two tailed t-test. Differences with a p-value of <0.05 were considered statistically significant. Experiments shown, except where indicated, are the means of multiple individual points from multiple separate experiments (±SEM). Statistical analyses were made using SigmaPlot and SigmaStat.

### Example 1

### Effects of Combined Exposure of Human Monocytic Leukemia Cells (U937) to UCN-01 and the MEK Inhibitor PD184352

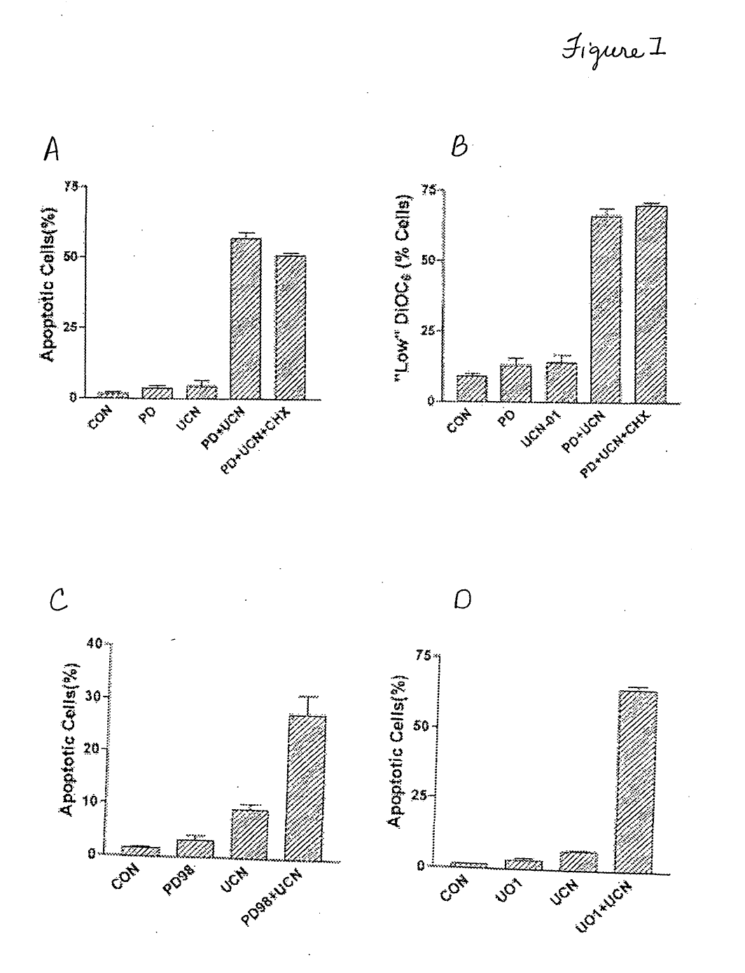

The effects of combined exposure of human monocytic leukemia cells (U937) to UCN-01 and the MEK inhibitor PD184352 were first examined in relation to MAPK activation and apoptosis. Unexpectedly, incubation with UCN-01 (150 nM) induced phosphorylation (activation) of MAPK by 2 h, and this effect persisted over the ensuing 18 h. Coincubation of U937 cells with PD184532 (10 mM) attenuated induction of phospho-MAPK at 2 h, and inhibition of MAPK activation was essentially complete after 18 h. To determine what impact this phenomenon had on cell fate, the extent of apoptosis was monitored in cells exposed to each agent individually and in combination. Whereas exposure to PD184352 or 150 nM UCN-01 alone was minimally toxic to these cells (10% apoptosis in each case), combined treatment resulted in a dramatic increase in cell death (i e., ;60%; FIG. 1A). Furthermore, this effect was not attenuated by coadministration of the protein synthesis inhibitor CHX (1 mM). Consistent with these findings, combined treatment with UCN-01 and PD184352, but not individual exposure, induced marked cleavage of procaspases-3 and -9, PARP degradation, and cytochrome c release into the cytoplasmic S-100 fraction. Cotreatment of cells with UCN-01 and PD184352 also resulted in a marked increase in the number of cells exhibiting loss of the mitochondrial membrane potential (e.g. ΔΨm ; FIG. 1B), an action that was also not attenuated by CHX. TUNEL assays confirmed that a small number of cells exposed to UCN-01 or PD 184352 alone for 18 h displayed DNA breaks containing overhanging 3′-OH ends, whereas coexposure resulted in a high percentage of TUNEL-positive cells. Similarly, agarose gel electrophoresis demonstrated a marked increase in oligonucleosomal DNA fragmentation in cells exposed to both agents.

Together, these findings indicate that coadministration of the MEK inhibitor PD184352 blocks MAPK activation and dramatically increases apoptosis in cancer cells exposed to a marginally toxic concentration of the cell cycle check point abrogator UCN-01.

### Example 2

### Extension to Other Known MEK1/2 Inhibitors

To determine whether these findings could be extended to other known MEK1/2 inhibitors, U937 cells were incubated for 24 h with 200 nM UCN-01 either alone or in combination with PD98059 (50 mM), an aminoflavone that was among the earliest of the MEK inhibitors to be investigated (Dudley, 1995), and U0126 (20 mM), the affinity of which for the CDK ATP-binding site is significantly greater than that of PD98059 (Favata et al. 1998). As noted in the case of PD184352, coadministration of minimally toxic concentrations of PD98059 or U0126 with 200 nM UCN-01 resulted in a marked potentiation of cell death, manifested by an increase in the morphological features of apoptosis (FIG. 1C and D), PARP degradation, and release of cytochrome c into the cytoplasm.

These findings demonstrate that multiple pharmacological compensatory cytoprotective pathway inhibitors are capable of substantially increasing the lethal actions of the cell cycle check point abrogator UCN-01 toward cancer cells.

### Example 3

### Generalization to Include Other Leukemia Cell Types

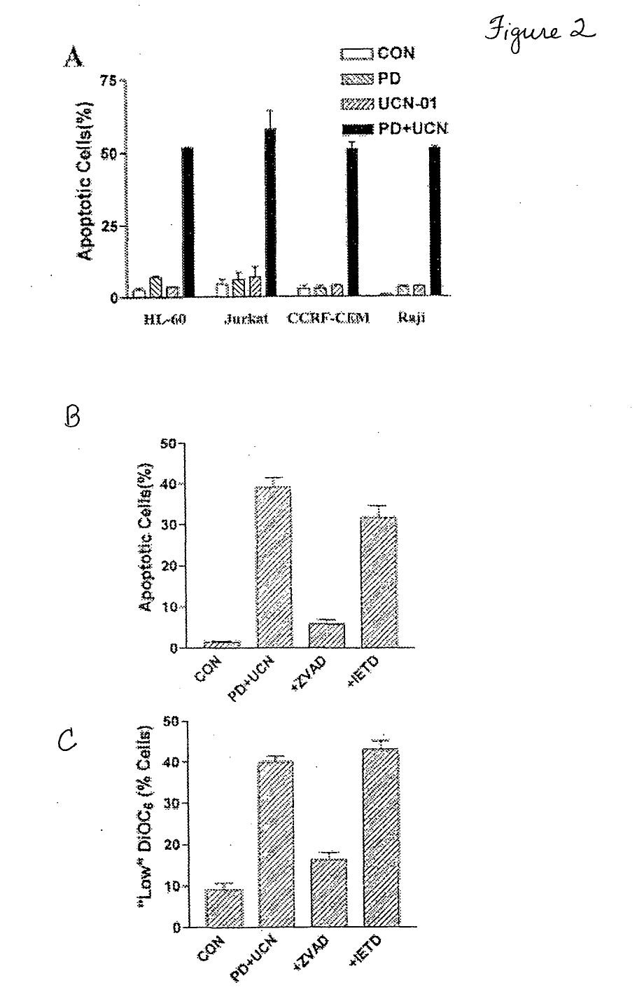

To establish whether the enhanced lethality of MEK inhibitors and UCN-01 was restricted to U937 cells or, instead, might be generalized to include other leukemia cell types, the effects of combined exposure to UCN-01 and PD184352 were examined in several. additional leukemia cell lines (FIG. 2). Because the sensitivity of these cells to UCN-01 differed somewhat from that of U937 cells, slightly higher UCN-01 concentrations (e.g., 150-300 nM) were used in some cases. On the basis of standard morphological criteria as well as evidence of PARP degradation, it was seen that combined treatment with UCN-01 and PD184352, administered at concentrations that were marginally toxic by themselves, resulted in a dramatic increase in cell death in HL-60 promyelocytic leukemia cells, T-lymphoblastic CCRF-CEM and Jurkat cells, and B-lymphoblastic lymphoma Raji cells (FIG. 2A). Qualitatively similar results were obtained when PD98059 and U0126 were used (data not shown). As in the case of U937 cells, UCN-01 treatment resulted in a substantial increase in MAPK activation in IL-60, CCRF-CEM, and in Jurkat cells; moreover, this effect was blocked by PD184352 (5 μM).

Thus, combined treatment with UCN-01 and MEK inhibitors prevented MAPK activation and produced a dramatic increase in apoptosis in a variety of myeloid and lymphoid cell lines, demonstrating that the coadministration of a cell cycle check point abrogation agent and an agent the inhibits a compensatory cytoprotective pathway is effective in promoting apoptosis in a wide variety of leukemia cell types.

### Example 4

### Investigation the Hierarchy of Events Accompanying Apoptosis

To investigate the hierarchy of events accompanying apoptosis induced by these agents, U937 cells were exposed to the combination of UCN-01 (150 nM) in conjunction with 10 μM PD184352 in the presence or absence of the broad caspase inhibitor ZVAD-fmk as well as the caspase-8 inhibitor IETD-fmk, after which cytochrome c release, loss of ΔΨm, caspase activation, PARP degradation, and apoptosis were monitored (FIG. 2, B and C).

Whereas ZVAD-fmk blocked induction of apoptosis and loss of ΔΨm in U937 cells exposed to UCN-01 and PD 184352, IETD was ineffective (FIG. 2, B and C). Similarly, ZVAD, but not IETD (20 μM each), prevented procaspase-3 cleavage and PARP degradation. In contrast, ZVAD, lile IETD, failed to reduce cytochrome c release in UCN-01/PD184352-treated cells.

These findings are compatible with the notion that cytochrome c release represents the primary event in UCN-01/PD184352-induced apoptosis, whereas the loss of mitochondrial membrane potential is a secondary process that stems from caspase activation. They also indicate that UCN-01/PD-induced apoptosis proceeds through a caspase 8-independent pathway.

### Example 5

### Contribution of PKC Inhibition to Pro-apoptotic Interactions with UCN-01

Because UCN-01 can act as an inhibitor of PKC (Mizuno, 1995), attempts were made to determine whether this action might be responsible for or contribute to pro-apoptotic interactions with UCN-01. To this end, U937 cells were exposed for 18 h to 10 mM PD 184352 alone or in combination with two known PKC inhibitors, ie., GFX (1 μM)or safingol (2 μM; Table 1). In separate studies, these drug concentrations were found to block PMA-mediated MAPK phosphorylation in U937 cells (data not shown). In marked contrast to interactions with UCN-01, coadministration of PD 184352 with either GFX or safingol produced relatively minor or no changes in apoptosis or loss of ΔΨm , arguing against the possibility that synergism between MEK inhibitors and UCN-01 solely involves PKC inhibition.

TABLE 1
Effects of combining PD184352 with the PKC inhibitors GFX or
safingol on apoptosis and loss of ΔΨm in U937 cells¹

| | Apoptotic cells (%) | “Low” DiOC₆ (% cells) |
|---|---|---|
| Control | 1.5 ± 0.3 | 11.7 ± 1.3 |
| GFX (1 μM) | 1.9 ± 0.2 | 13.2 ± 0.6 |
| PD184352 (10 μM) | 2.1 ± 0.1 | 14.7 ± 1.0 |
| GFX + PD | 2.2 ± 0.2 | 15.7 ± 1.7 |
| Control | 1.5 ± 0.2 | 10.5 ± 0.2 |
| Safingol (2 μM) | 1.9 ± 0.2 | 13.6 ± 0.8 |
| PD184352 (10 μM) | 1.8 ± 0.1 | 12.1 ± 0.2 |
| Safingol + PD | 2.4 ± 0.1 | 16.4 ± 1.1 |

¹Logarithmically growing U937 cells were exposed to PD184352 (10 μM) ± safingol (2 μM) or GFX (1 μM) for 18 h, after which the percentage of morphologically apoptotic cells or the fraction of cells displaying reduced uptake of DiOC 6 was determined as described previously. Values represent the means ± SD for three separate experiments performed in triplicate.

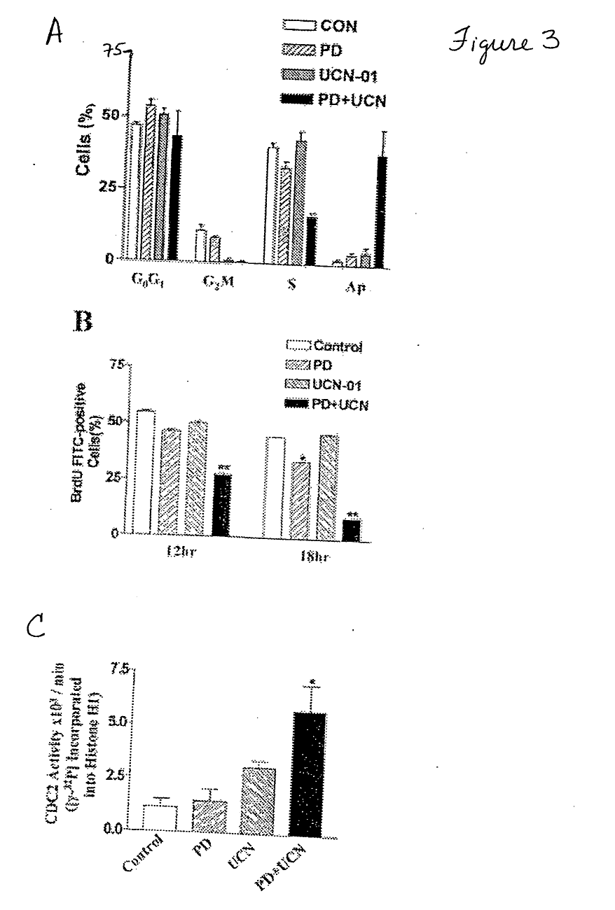

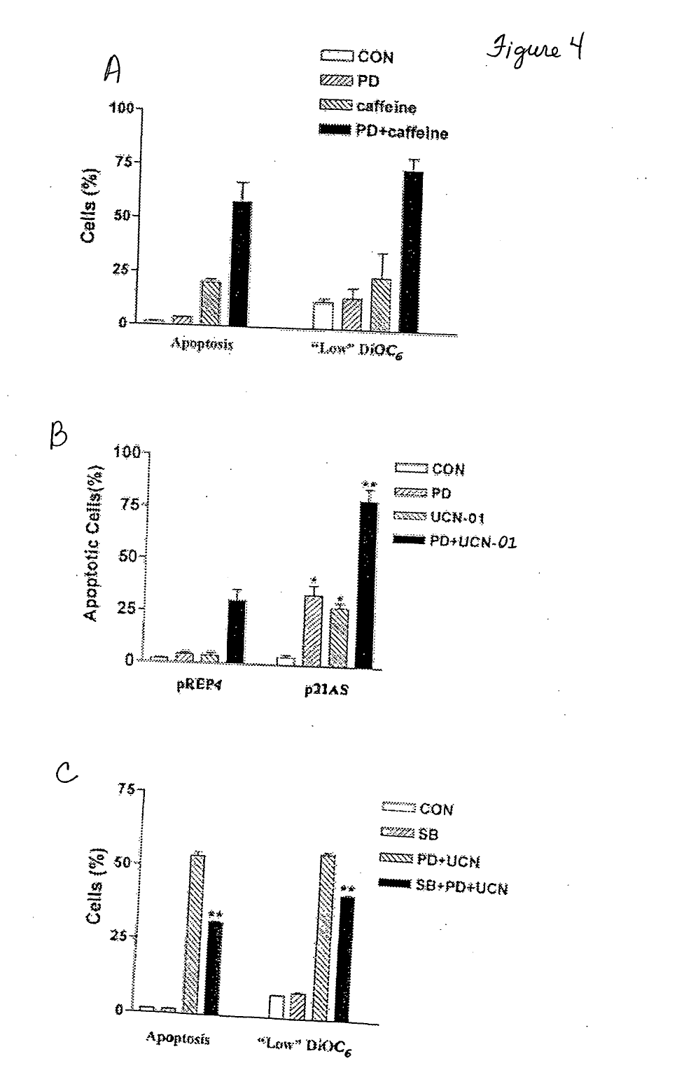

Interactions between PD184352 and UCN-01 were then examined in relation to cell cycle events (FIG. 3). Administration of PD184352 by itself for 18 h had little effect on the cell cycle distribution of U937 cells, whereas UCN-01 (150 nM) primarily depleted the G2 M population (FIG. 3A). When the agents were combined, elimination of the G2 M fraction persisted, an event accompanied by a significant decline in the S-phase population and corresponding increase in the subdiploid apoptotic fraction. Examination of BrdUrd incorporation, reflecting DNA synthesis, at 12 and 18 h of drug exposure revealed a modest decline in the number of BrdUrd-positive cells after PD184352 treatment, but no effect after incubation with UCN-01 (FIG. 3B). However, a very substantial decline in BrdUrd-positive cells was noted 12 h and particularly 18 h after PD184352/UCN-01 exposure. Consistent with its inhibitory effects on Chk (Graves, et al., 2000), UCN-01 modestly reduced the amount of cdc25C phosphatase coimmunoprecipitating with 14-3-3 proteins. However, this effect was more pronounced in cells treated with both UCN-01 and PD184352. Moreover, cells exposed to the combination of UCN-01 and PD184352 for 18 h exhibited the greatest diminution in phosphorylation of p34 cdc2 on tyrosine15. In contrast, total expression of p34 cdc2 was essentially unchanged. Activity of p34 cdc2, reflected by phosphorylation of histone H1, was greater in cells exposed to the combination of UCN-01 and PD184352 than in those exposed to UCN-01 alone at both the 14-h and 18-h intervals. Lastly, quantification of p34 cdc2 activity at 18 h by kinase assay confirmed enhanced activation in cells exposed to UCN-01±PD184352 compared with values obtained for UCN-01 alone (FIG. 3C). Interestingly, caffeine (2 mM; 18 h), an inhibitor of ATM acting upstream of Chk1 (Blasina et al., 1999), also reduced binding of cdc2 to 14-3-3 proteins and, when combined with PD184352, markedly decreased cdc2 phosphorylation. Caffeine also substantially increased apoptosis and mitochondrial damage in PD184352-treated cells (FIG. 4A).

Together, these findings raise the possibility that interactions between PD184352 and UCN-01 may involve interference with checkpoint function and, as a consequence, inappropriate (i.e., unscheduled) activation of p34 cdc2.

### Example 6

### Identification of Downstream Targets Responsible for Enhanced Apoptosis

To identify downstream targets of p42/44 MAPK that might be responsible for or contribute to enhanced apoptosis in UCN-01/PD184352-treated cells, the effects of these agents were examined with respect to expression of p21^{CIP1}, p27^{KIP1}, and CREB, each of which has been linked to antiapoptotic actions (St. Croix et al., 1996; Bonni et al., 1999; Wang et al., 1998). It was observed that PMA (5 nM; 24 h) robustly increased p21^{CIP1} expression, whereas UCN-01, either alone or in combination with PD184352, had no discernible effect. Similarly, constitutive expression of p27^{KIP1} in U937 cells was not altered by either drug alone or in combination. However, a clear reduction in expression of phosphorylated CREB was noted in cells exposed to the UCN-01/PD184352 combination.

These findings raise the possibility that interference with the downstream MAPK cytoprotective target CREB may contribute to potentiation of apoptosis in UCN-01/PD184352-treated cells.

Studies were also performed in U937 cells stably expressing a p21^{CIP1} antisense construct that are impaired in their capacity to up-regulate p21^{CIP1} in response to PMA (Wang et al. 1998) and are more sensitive than wild-type cells to apoptosis induced by agents such as 1-b-D-arabinofulranosylcytosine (Wang et al. 1999). Dysregulation of p21^{CIP1} resulted in a modest but significant increase in apoptosis in cells exposed to UCN-01 or PD184352 individually; moreover, the combination of these agents was significantly more lethal to p21^{CIP1} antisense-expressing cells (FIG. 4B). Similar results were observed with PD98059 (data not shown).

Because p21^{CIP1} expression is already dysregulated in the antisense line, these and the preceding findings argue against the possibility that potentiation of UCN-01-related apoptosis by MEK/MAPK inhibitors involves impaired induction of the downstream MAPK target p21^{CIP1}.

### Example 7

### Assessment of the Influence of MEK Inhibitors and UCN-01 on Other MAPK Pathways

To assess the influence of MEK inhibitors and UCN-01 on other MAPK pathways, the effects of these agents were examined in relation to JNK and p38 phosphorylation. In contrast to the increase in expression of phospho-MAPK, UCN-01, either alone or in combination with PD184352, did not noticeably induce JNK phos-pholylation in U937 cells. Similar results were obtained with PD98059 (data not shown). In separate studies (Freemerman et al., 1996) involving U937 cell transfectants, stable expression of a dominant-negative c-Jun transactivation domain-deficient mutant (TAM67) did not attenuate PD184352/UCN-01-mediated apoptosis (data not shown). Interestingly, coadministration of UCN-01 and PD184352, but not individual drug exposure, resulted in a marked increase in expression of phospho-p38 MAPK. However, coadministration of the p38 MAPK inhibitor SB203580 (10 μM) only partially attenuated apoptosis and mito-chondrial injury in PD184352/UCN-01-treated cells (FIG. 4C). Lastly, combined drug exposure exerted did not increase expression of the MKP1 and MKP3 phosphatases.

Together, these findings indicate that potentiation of UCN-01-related apoptosis by MEK inhibitors is accompanied by a marked increase in p38 MAPK but not JNK phosphorylation.

### Example 8

### Determination of Whether Coadministration of UCN-01 and MEK Inhibitors Modifies Expression of Apoptotic Regulatory Proteins

To determine whether coadministration UCN-01 and MEK inhibitors modified the expression of apoptotic regulatory proteins, levels of Bcl-2, Bcl-X_{L}, Bax, and XIAP were monitored by Western analysis. Coadministration of UCN-01 and PD184352 did not result in a significant change in expression of Bcl-2, Bcl-X_{L}, Bax, or XIAP. Similar results were observed in cells exposed to the combination of UCN-01 and PD98059 (data not shown). In addition, separation of proteins on a 15-cm, 12% SDS-PAGE gel, which permitted visualization of a slowly migrating, putatively phosphorylated Bcl-2 species revealed no significant change after exposure of cells to PD184352 in combination with UCN-01.

These observations argue against the possibility that potentiation of UCN-01-induced apoptosis by MEK inhibitors stemmed from increased expression of Bax or diminished expression of the antiapoptotic proteins Bcl-2, Bcl-X_{L}, or XIAP.

### Example 9

### Impact of Combined Treatment of U937 Cells with UCN-01 and PD184352 on Clonogenic Survival

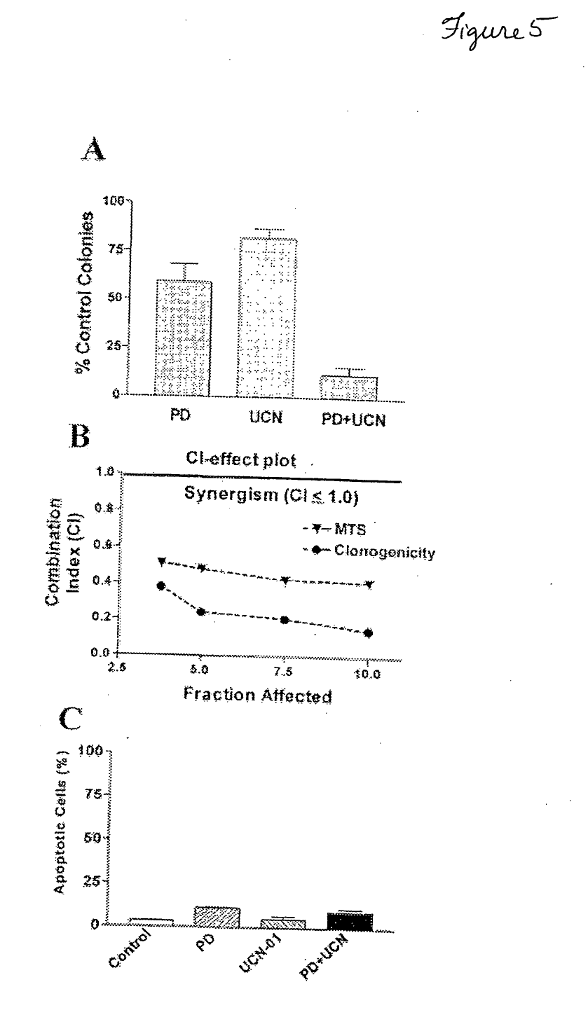

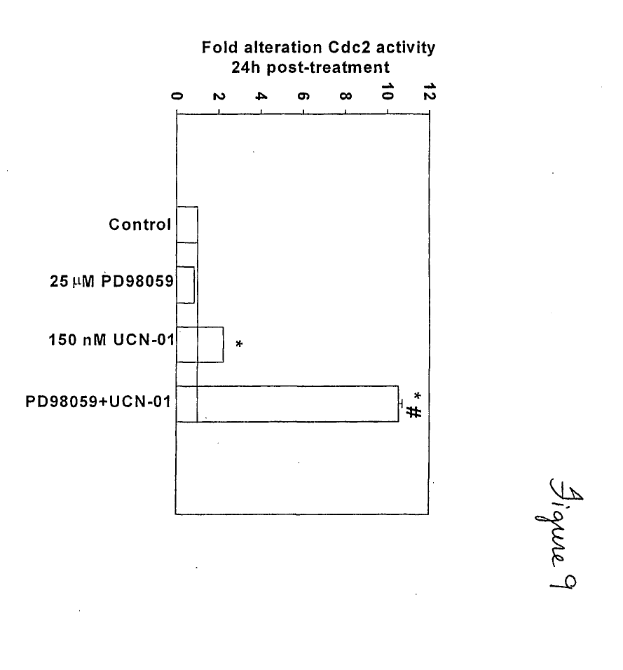

The impact of combined treatment of U937 cells with UCN-01 and PD184352 was examined in relation to effects on clonogenic survival (FIG. 5). UCN-01 (150 nM; 18 h) by itself had a very modest effect on colony formation, whereas PD184352 (10 μM;18 h) administered alone reduced clonogenic survival by 40%. However, combined treatment with both agents resulted in a substantial reduction in clonogenicity (e.g., to 10% of control values; FIG. 5A). Furthermore, median dose effect analysis (Chou and Talalay, 1984) was used to characterize interactions between these agents, administered at a fixed ratio (e.g., PD/UCN-01, 50:1), over a range of UCN-01 concentrations (e.g., 75-200 nM; FIG. 9B). Combination index values for the drug combination, using either a reduction in clonogenicity (●) or loss of viability by MTS assay (▾) as end points, were considerably less than 1.0 (FIG. 5B), corresponding to a highly synergistic interaction.

These findings indicate that enhanced apoptosis in cells exposed to UCN-01 in combination with a MEK/MAPK inhibitor is accompanied by a significant reduction in leukemic cell viability and self-renewal capacity.

Finally, parallel studies were carried out using normal peripheral blood mononuclear cells. Exposure of such cells for 24 h to 150 nM UCN-01±10 μM PD184352 did not result in a significant increase in apoptosis for any of the conditions (e.g. ,5% increases versus controls; P≧0.05 for each condition). Similar results were observed in cells exposed to the combination of UCN-01 and PD98059 or U0126 (data not shown).

These findings raise the possibility that coadministration of UCN-01 with MEK/MAPK inhibitors may not represent a potent apoptotic stimulus in at least some normal hematopoietic cells.

### Example 10

### Treatment of Carcinoma Cells with UCN-01 Activates the MAPK Pathway

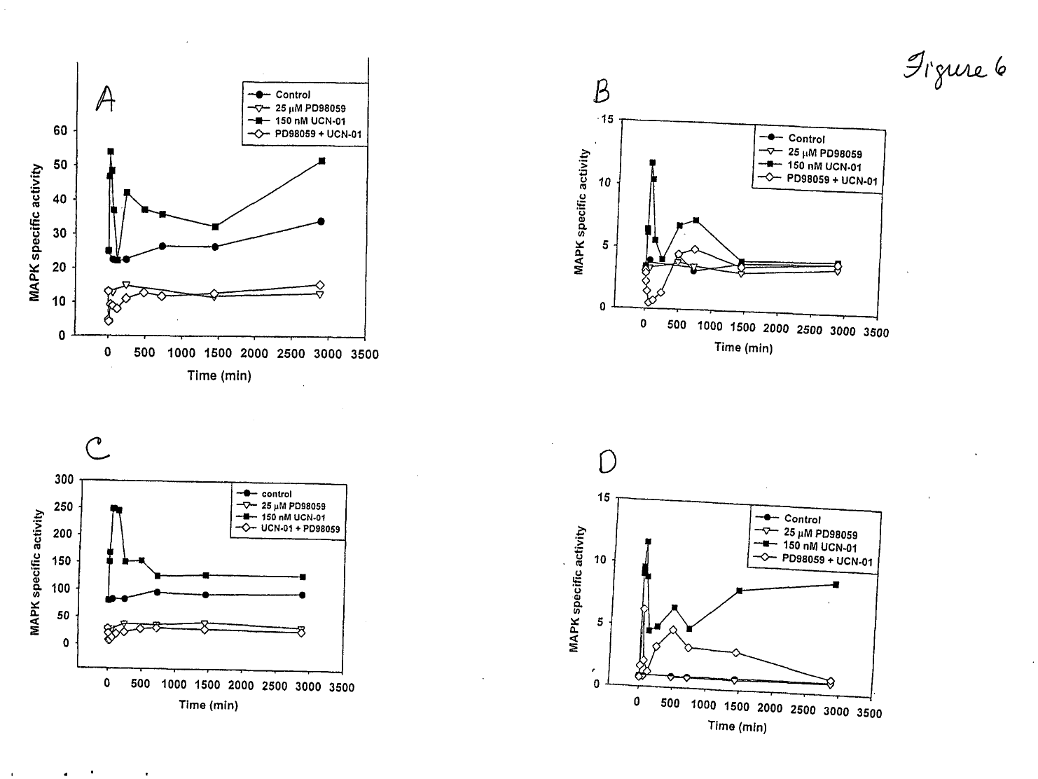

UCN-01 is a known PKC and Chk1 inhibitor. Based on its capacity to inhibit PKC isoforms, we postulated that UCN-01 would suppress the activity of signaling pathways downstream of PKC, including the MAPK pathway. However, when MCF7, MDA-MB-231, T47D and DU145 cells were treated with a clinically relevant dose of UCN-01 (150 nM), activation of MAPK/ERK was observed in immune complex kinase assays that was variably prolonged for intervals of 12 h to 24 h.(FIG. 6A-D). MAPK activation by UCN-01 was opposed by the MEK1/2 inhibitor PD98059 (25 μM) (FIG. 6A-D), and by other structurally unrelated MEK1/2 inhibitors PD184352 (10 μM) and U0126 (5 μM) (data not shown). In these studies, little modulation of JNK/SAPK or p38 MAPK pathway activity was observed following treatment with the drugs (data not shown).

MAPK activation correlated with enhanced phosphorylation of both MEK1/2 (S218/S222) and ERK1/2 (T183/Y185) as judged by immunoblotting of cell lysates. Of note, while the enhanced decrease in ERK1/2 immune complex kinase activity in cells treated with UCN-01 and MEK1/2 inhibitor was reflected in a parallel loss of ERK1/2 phosphorylation, no additional loss of MEK1/2 phosphorylation or MEK1/2 activity was observed under these conditions (data not shown). This finding argues that under conditions of the drug combination, either MEK1/2 activity is being suppressed independently of “Raf”-mediated S217/S221 phosphorylation or that a MAPK/ERK phosphatase is becoming activated.

### Example 11

### Combined Treatment of Carcinoma Cells with UCN-01 and MEK1/2 Inhibitor Reduces Proliferative Potential and Increases Cell Killing Within 24 h

Because the activity of the MAPK pathway was reduced following drug treatment, we next investigated the impact of reduced pathway function on tumor cell proliferation. Individual treatment of cells with either MEK1/2 inhibitor or UCN-01 alone reduced the proliferative capacity of carcinoma cells, reflected by MTT assays, to varying degrees, however, the proliferation of cells exposed to the combination of MEK1/2 inhibitor and UCN-01 was significantly lower (>80% in all cell types) than either treatment alone.

Because exposure of cells to MEK1/2 inhibitor and UCN-01 reduced proliferative capacity, attempts were made to determine whether this effect reflected reduced cell viability. To assess the caspase-dependence of these events, cells were incubated with the pan-caspase inhibitor ZVAD. Furthermore, since both UCN-01 and MEK1/2 inhibitors are known radio-sensitizers, studies were also performed in parallel using ionizing radiation. Cells were exposed to UCN-01 and MEK1/2 inhibitors (±radiation; 2 Gy), after which cell viability was determined 24 h following treatment by monitoring double stranded DNA breaks, as well as by 7AAD and nuclear morphology (not shown).

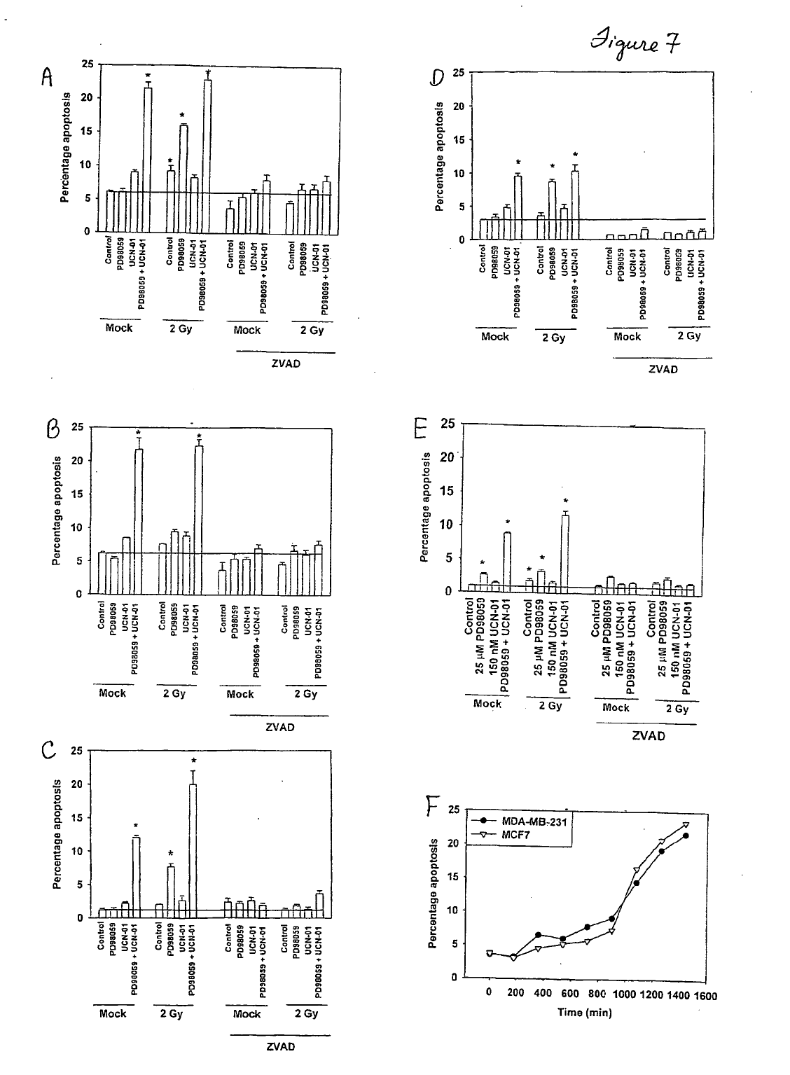

In all cell types examined neither MEK1/2 inhibition, UCN-01 treatment, nor radiation exposure alone induced substantial reductions in cell viability within 24 h. However, when cells were exposed to MEK1/2 inhibitor and UCN-01, a significant potentiation of apoptosis was observed after 24 h that was abolished by the pan-caspase inhibitor ZVAD (FIG. 7). In all cell types examined, with the exception of DU145 cells which are null for the pro-apoptotic protein BAX, radiation exposure did not result in a further significant reduction in viability of cells exposed to the combination of UCN-01 and MEK1/2 inhibitor (FIG. 7C).

In contrast to these malignant cells, primary human mammary epithelial cells, primary mouse hepatocytes, normal human peripheral blood mononuclear cells, or a purified population of human CD34⁺ stem cells did not exhibit an enhancement in apoptosis following exposure to UCN-01 and MEK1/2 inhibitor (data not shown). Also, notably, the PKC inhibitor bisindolylmaleimide (1 μM) did not mimic the actions of UCN-01, consistent with a PKC-independent mechanism of action for UNC-01 (data not shown). Collectively, these findings argue that the reduction in proliferation of cells exposed to UCN-01 and MEK1/2 inhibitors reflects, at least in part, potentiation of caspase-dependent apoptosis.

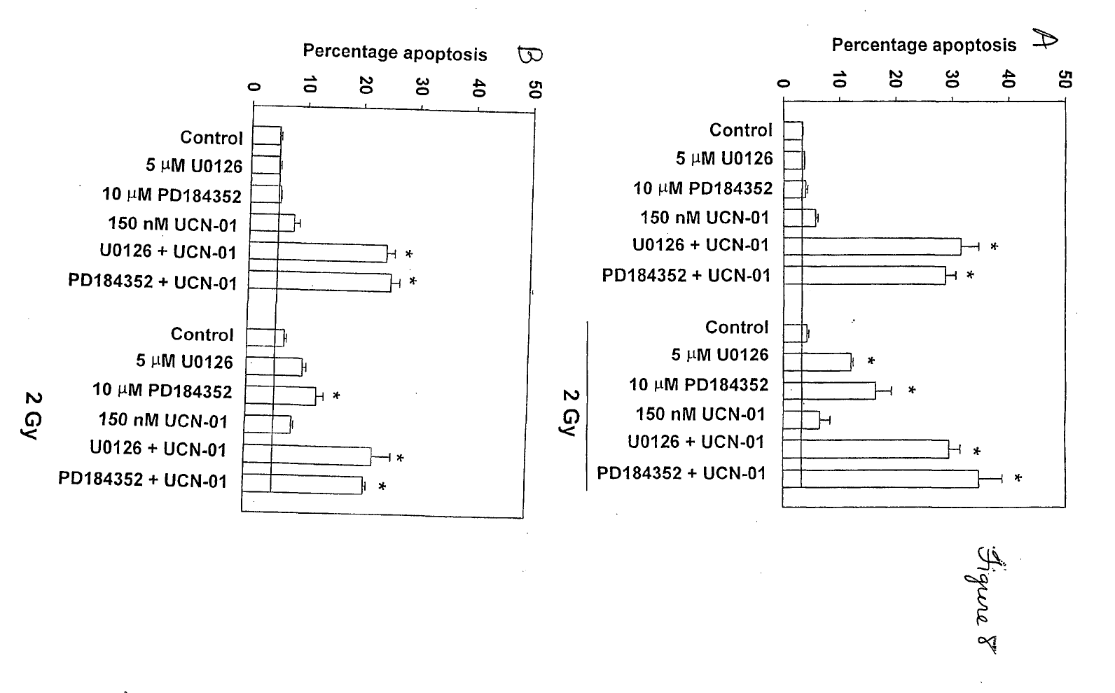

In addition to monitoring apoptosis 24 h after exposure, studies were also performed to characterize the ability of UCN-01/MEK1/2 inhibitor treatment to potentiate apoptosis over a 24 h time course. Combined inhibitor treatment weakly enhanced apoptosis in MDA-MB-231 and MCF7 cells 6 h after exposure, with only a doubling in the basal percentage of dead cells, from ˜3% to ˜6% (FIG. 7F). However, it was only after an exposure interval of 18-24 h that ≧15-25% of total cells were classified as apoptotic based upon evidence of DNA strand breakage (FIGS. 2A and 2B cf FIG. 7F). Of note, epithelial cancer cells did not undergo significant apoptosis until 18-24 h, whereas their hematopoietic counterparts were found in parallel studies to become apoptotic more rapidly, within 14-18 h [Dai et al., 2001]. This finding suggests the mechanism of cell Idiling may be different comparing solid and liquid tumor cells. The potentiation of apoptosis was not limited to the MEK1/2 inhibitor PD98059, as we found that the chemically dissimilar MEK1/2 inhibitors PD184352 and U0126 also enhanced apoptosis in combination with UCN-01 (FIG. 8).

These findings demonstrate that the coadministration of a cell cycle check point abrogator and an inhibitor of a compensatory cytoprotective pathway (many examples of which were tested) is effective in promoting apoptosis in a variety of cancer cell types.

### Example 12

### Combined Treatment of Carcinoma Cells with UCN-01 and MEK1/2 Inhibitor Increases Release of Cytochrome c Into the Cytosol and Correlates With Reduced Cdc2 Y15 Phosphorylation and With Reduced Expression of p21^{Cip-1/WAF1/mda6}

In view of evidence that combined exposure to UCN-01 and MEK1/2 inhibitors promoted cell death in epithelial carcinoma cells, the effects of co-treatment with these agents was examined in relation to various events implicated in the apoptotic process, including mitochondrial damage, pro-caspase cleavage/activation, and perturbations in various cell cycle regulatory proteins. Combined exposure of MDA-MB-231 carcinoma cells to a MEK1/2 inhibitor and UCN-01, but not individual treatment, induced cleavage of pro-caspases -8, -9 and -3. Notably, cleavage of pro-caspase 9, and to a lesser extent pro-caspase 3, occurred 6-12 h prior to cleavage of pro-caspase 8. Combined exposure of MDA-MB-231 cells to a MEK1/2 inhibitor and UCN-01, but not individual treatment, induced a weak reduction in Cdc2 Y15 phosphorylation that correlated with cleavage of pro-caspases 9 and 3. In contrast, cleavage of the pro-apoptotic protein BID, a profound reduction in Cdc2 Y15 phosphorylation, enhanced Cdc2 activity and reduced 14-3-3 protein association with Cdc25C correlated temporally with the delayed 18-24 h cleavage of pro-caspase 8 (FIGS. 9, not shown) and with the large increase in apoptosis observed at 18-24 h post-treatment (FIG. 7F). Similar data for pro-caspase cleavage were obtained in DU145 cells (not shown). Cleavage of pro-caspases 8, 9, 6 and 7 was observed in MCF7 cells which lack expression of pro-caspase 3 (not shown).

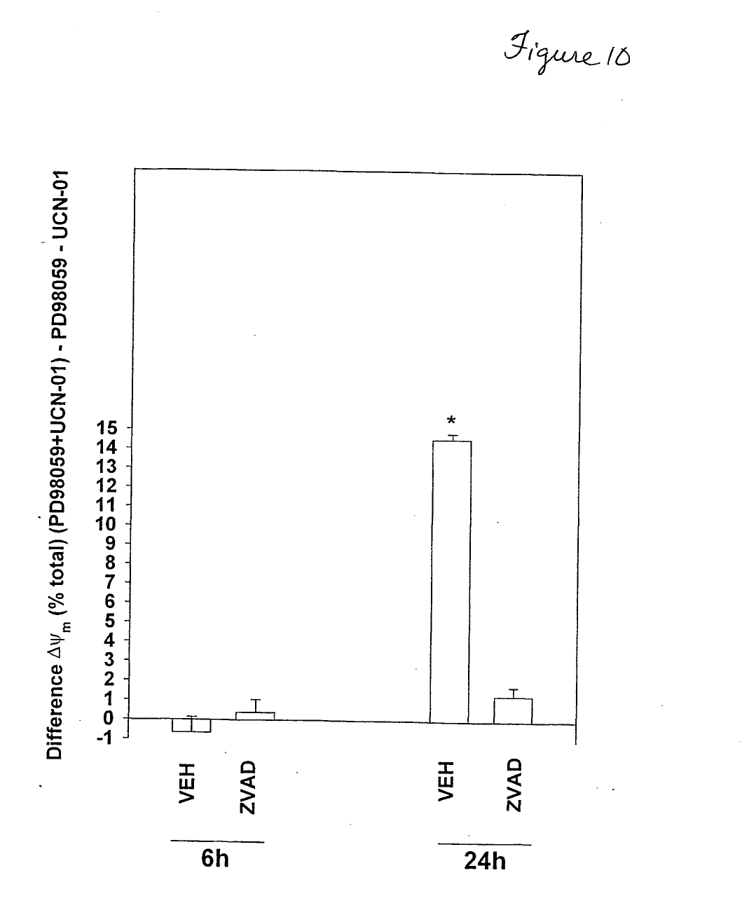

Co-treatment of MDA-MB-231 cells with UCN-01 and MEK1/2 inhibitor also resulted in a marked increase in the number of cells exhibiting loss of the mitochondrial membrane potential, 24 h, but not 6 h, after exposure to the drugs (i.e., Δψm; FIG. 10). In contrast, release of cytochrome c into the cytosol was observed at both 6 h and 24 h after exposure. Similar data were also obtained in DU145 cells (data not shown). Treatment of cells with a pan-caspase inhibitor ZVAD failed to reduce cytochrome c release in UCN-01/MEK1/2 inhibitor-treated cells 6 h after exposure but prevented loss of Δψₘ at 24 h. These findings are compatible with the concept that cytochrome c release represents the primary event in the apoptotic response of these cells to UCN-01/MEK1/2 inhibitor treatment, whereas the loss of Δψₘ is a secondary process stemming from subsequent activation of pro-caspases, including pro-caspase 8, and the cleavage of facilitator proteins, i.e., BID.

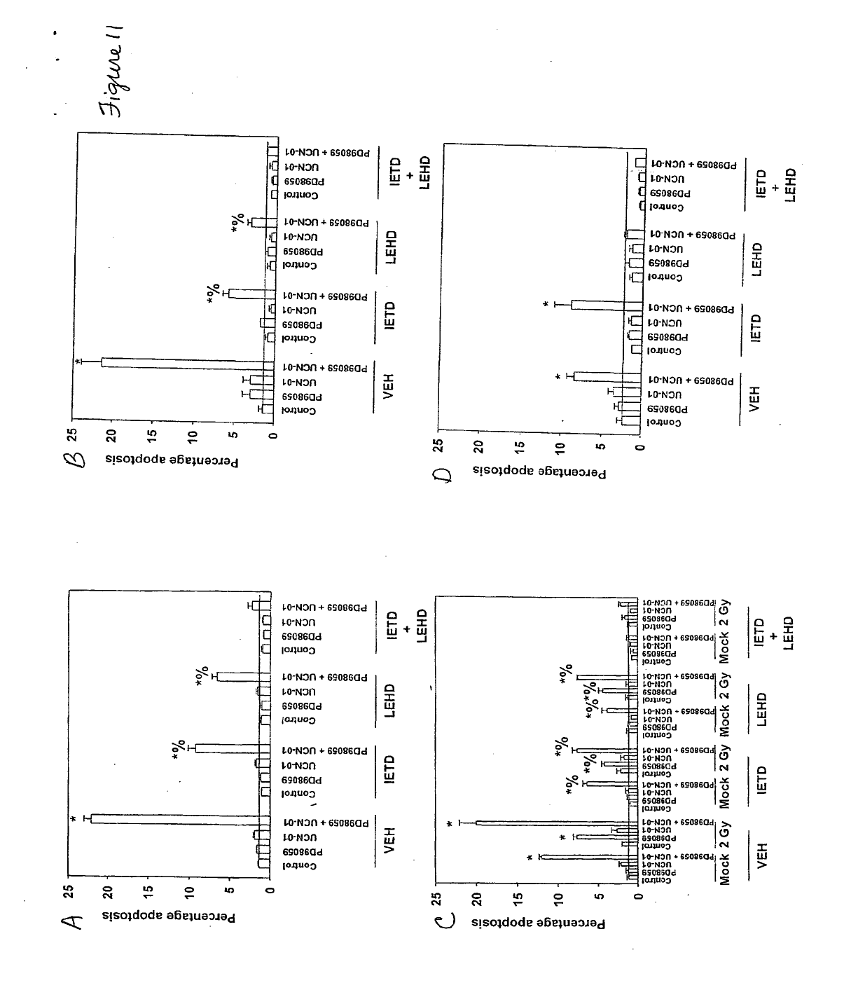

Attempts were then made to determine whether peptide inhibitors specific for caspase 9 (LEHD-fmk) and for caspase 8 (IETD-fmk) could block the apoptotic response following combined treatment with MEK1/2 inhibitor and UCN-01. Incubation of cells individually with either LEHD-fmk or IETD-fmk partially attenuated the potentiation of apoptosis following treatment of carcinoma cells with MEK1/2 inhibitor and UCN-01 (FIG. 11A-D). Treatment with both inhibitors was required to abolish the apoptotic response. Thus in contrast to hematopoietic cells [Dai et al., 2001], incubation of epithelial carcinoma cells with both a caspase 9 (LEHD-fmk) and a caspase 8 (IETD-fmk) inhibitor was required for complete inhibition of the potentiation of apoptosis induced by MEK1/2 inhibitor and UCN-01 treatment.

### Example 14

### Over-Expression of Bxl-_{XL} Blocks Potentiation of Apoptosis Caused by Combined Treatment with MEK1/2 Inhibitor and UCN-01

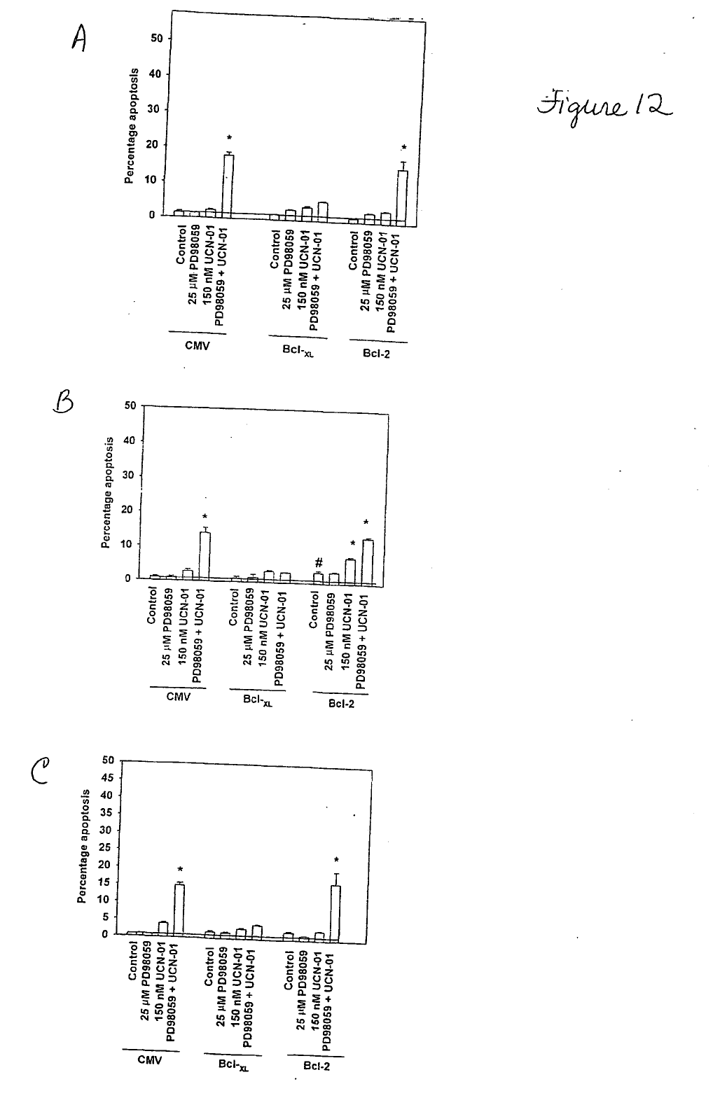

In view of evidence that activation of pro-caspase-9 was involved in the enhanced apoptosis observed in cells exposed to UCN-01 and MEK1/2 inhibitors, an effort was made to determine whether over-expression of the mitochondrial anti-apoptotic proteins Bcl-X_{L} and Bcl-2 protected carcinoma cells from drug-induced apoptosis. Cells were infected with recombinant adenoviruses to express either an empty vector, Bcl-2, or Bcl-X_{L}. Increased expression of Bcl-X_{L} abolished the potentiation of apoptosis induced by combined treatment with PD98059 and UCN-01 (FIG. 12A-C). Contrary to expectations, over-expression of Bcl-2 did not protect cells from increased cell killing, consistent with the results of previous studies suggesting a differential cytoprotective effect of these anti-apoptotic proteins [Lebedeva et al., 2000; Luo et al., 2000]. Over-expression of Bcl-_{XL} abolished the release of cytochrome c into the cytosol of MDA-MB-231 cells, but did not alter the drug-stimulated increase in mitochondrial BAX levels. Collectively, these findings further argue that enhanced apoptosis in cells exposed to UCN-01/MEK1/2 inhibitors proceeds through a cytochrome c-dependent mechanism that is blocked by Bcl-X_{L} over-expression.

### Example 15

### Potentiation of Apoptosis Correlates With Reduced Cell Numbers in S Phase and G2/M Phase

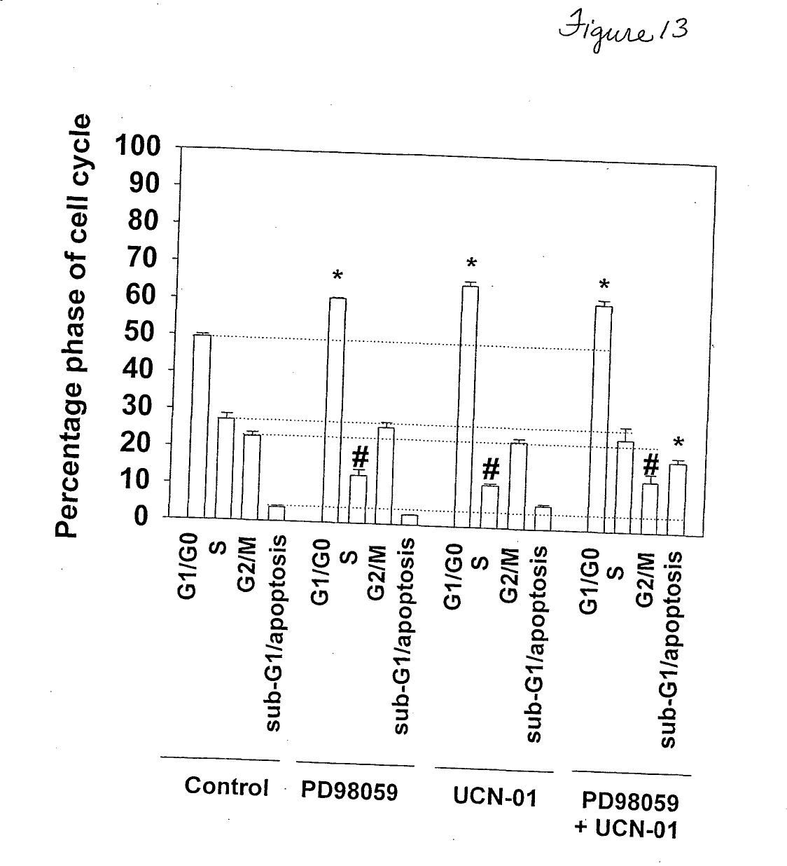

Previous studies from several groups have demonstrated that enhanced MAPK signaling is important for cell cycle progression through G2/M phase after drug- or radiation-induced cell cycle arrest. Thus, interactions between PD98059 and UCN-01 were examined in relation to cell cycle events (FIG. 13). Administration of either a MEK1/2 inhibitor or UCN-01 individually for 24 hr enhanced cell numbers in G1 phase and primarily depleted the S phase population. When the agents were combined, a substantial reduction in the G2/M fraction occurred, an event accompanied by a significant increase in the S-phase population and corresponding increase in the sub-diploid apoptotic fraction. Of note, only exposure to the combination of UCN-01 and PD98059, but not the drugs individually, exhibited a pronounced reduction in phosphorylation of Cdc2 on tyrosine 15. Together, these findings raise the possibility that interactions between PD98059 and UCN-01 may interfere with checkpoint function and cause unscheduled Cdc2 activation.

To identify other potential downstream targets of MAPK that might be responsible for, or contribute to, the enhancement of apoptosis in UCN-01/PD98059-treated cells, the effects of these agents were also examined with respect to expression of the Cdk inhibitor protein p21^{Cip-1/WAF1/mda6} (p21), which has been linked to growth arrest and anti-apoptotic actions. Treatment of MDA-MB-231 cells with UCN-01 caused prolonged MAPK activation and increased expression of p21, 6-24 h following treatment, which was reduced to basal control levels by PD98059. Previous findings by many groups have linked prolonged intense MAPK signaling to p53-independent increases in the expression of p21 [Park et al., 2000]. Similar data were obtained in MCF7, T47D and DU145 cells (data not shown). These findings, together with the previous data, raise the possibility that interference with p21 expression and Cdc2 phosphorylation may contribute to potentiation of apoptosis in UCN-01/PD98059-treated cells.

### Example 16

### Combined Exposure to MEK1/2 Inhibitor and UCN-01 for 48 h Diminishes Clonogenic Survival of Carcinoma Cells Which is Reduced Further by Radiation Exposure

Cells were treated with UCN-01, PD98059 or radiation in various combinations. Cells were re-plated 48 h after treatment and the impact of combined treatment of carcinoma cells with UCN-01 and PD98059 was examined in relation to effects on clonogenic survival (Table 1). Treatment with either UCN-01 or PD98059 by themselves generally had very modest effects on subsequent colony formation. However, combined treatment of cells with both agents resulted in a substantial reduction in clonogenicity (Table 1). These findings indicate that enhanced apoptosis in cells exposed to UCN-01 in combination with a MEK1/2 inhibitor is accompanied by a subsequent significant reduction in carcinoma cell viability and self-renewal capacity.

Of note, while UCN-01/MEK1/2 inhibitor-induced apoptosis was not further enhanced following radiation exposure as noted above (Example 11), the results presented in this Example show that irradiation of drug-treated cells resulted in a significant, marked reduction in clonogenic survival of cancer cells. This effect was pronounced in DU145, T47D and MDA-MB-231 cells. MCF7 cells, expressing wild type p53, were highly sensitive to the drug combination, and this effect was increased in an additive fashion by radiation. These data suggest that radiation enhances cell killing in the presence of the drug combination through a non-apoptotic mechanism. Further, these observations indicate that the co-administration of a cell cycle checkpoint abrogation agent and an agent that inhibits a compensatory cytoprotective pathway provides a means to sensitize cancer cells to the effects of radiation.

TABLE 1
Combined exposure of carcinoma cells to UCN-01 and MEK1/2 inhibitors
reduces clonogenic survival and is enhanced by radiation exposure.¹

| Cell | | | | | 2 Gy | 2 Gy | 2 Gy | 2 Gy PD + |
|---|---|---|---|---|---|---|---|---|
| Type | Control | PD98059 | UCN-01 | PD + U | control | PD98059 | UCN-01 | U |
| DU145 | 100 ± 1 | 99 ± 6 | 82 ± 4 | 55 ± 1* | 67 ± 3 | 65 ± 2 | 68 ± 2 | 21 ± 1*% |
| MCF7 | 100 ± 1 | 91 ± 5 | 69 ± 6 | 25 ± 2* | 61 ± 4 | 39 ± 3# | 44 ± 3 | 16 ± 4*% |
| T47D | 100 ± 4 | 97 ± 2 | 103 ± 8 | 62 ± 1* | 83 ± 3 | 69 ± 5 | 43 b ± 4 | 42 ± 4% |
| MDA | 100 ± 5 | 91 ± 1 | 97 ± 3 | 43 ± 1* | 63 ± 3 | 44 ± 1# | 55 ± 2 | 10 ± 1*% |
| ¹Cells were incubated with matched vehicle control (DMSO), with 25 μM PD98059 alone, with 150 nM UCN-01 alone, or with 25 μM PD98059 and 150 nM UCN-01. After 30 min, cells were either exposed to radiation (2 Gy) or mock irradiated. Identical portions of viable cells were taken 48 hours post irradiation and replated on Linbro plates at densities of 500 cells/well and 1000 cells/well. Cells were incubated for 10-14 days after which they were stained and | | | | | | | | |
| # colony number determined. A colony was defined as a cluster of 50 or more cells. MDA-MB-231 cells are referred to in the Table as MDA. Data are normalized to the plating efficiency of control unirradiated cells which is defined as 100% (Data shown are the number of staining colonies, 3-5 parallel individual experiments ± SEM) which were examined and counted via light microscopy. | | | | | | | | |

*p < 0.05 less than control value;
#p < 0.05 less than irradiated value without MAPK inhibition;
%p < 0.05 less than corresponding unirradiated value.

### Example 17

### Treatment of Human Leukemia Cells With UCN-01 in Combination With a PI3K Pathway Inhibitor, LY294002

In order to confirm that inhibitors of compensatory pathways other than the MEK 1/2 pathway would also induce apoptosis in cancer cells in combination with a cell cycle checkpoint abrogation agent, the combination of LY294002 (a PI3K pathway inhibitor) and

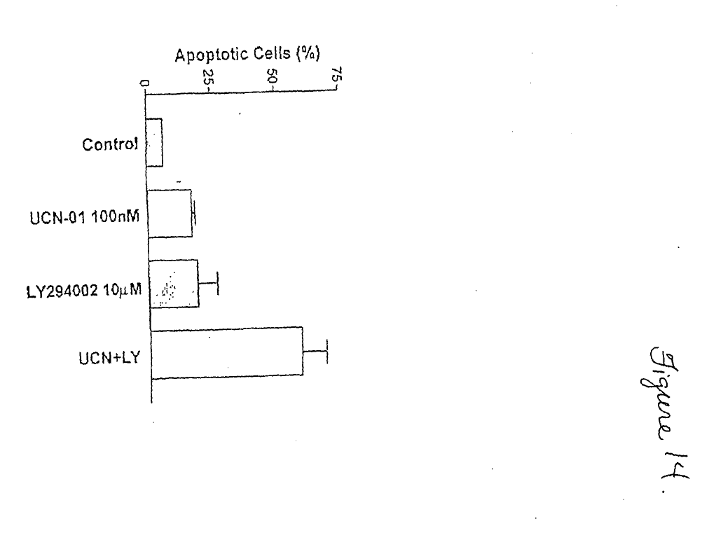

UCN-01 was assayed. U937 human leukemia cells were subjected to treatment with the two agents and the results are given in FIG. 14. As can be seen, the percentage of apoptotic cells increased dramatically from about 20% when either agent was administered alone, to about 60% when the two agents were co-administered.. These findings show that simultaneous checkpoint abrogation and inhibition of the PI3K pathway, as in the case of the MEK1/2 pathway, causes a significant increase in the level of apoptosis observed in human cancer cells.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

References

Alinaga, S., Nomura, K., Gomi, K., and Okabe, M. Enhancement of antitumor activity of mitomycin C in vitro and in vivo by UCN-01, a selective inhibitor of protein kinase C. Cancer Chemother. Pharmacol., 32: 183-189, 1993.

Alinaga, S., Nomura, K., Gomi, K., and Okabe, M. Effect of UCN-01, a selective inhibitor of protein kinase C, on the cell-cycle distributor of human epidermoid carcinoma, A431 cells. Cancer Chemother. Pharmacol., 33: 273-280, 1994.

Blasina, A., Price, B. D., Turenne, G. A., and McGowan, C. H. Caffeine inhibits the checkpoint kinase ATM. Curr. Biol., 9: 1135-1138, 1999.

Bonni, A., Brunet, A., West, A. E., Datta, S. R., Takasu, M. A., and Greenberg, M. E. Cell survival promoted by the Ras-MAPK signaling pathway by transcription-dependent and independent mechanisms. Science (Wash. D.C.), 286: 1358-1362, 1999.

Bunch, R. T., and Eastman, A. Enhancement of cis-platinum-induced cytotoxicity by 7-hydroxystaurosporine, a new G2 checkpoint inhibitor. Clin. Cancer Res., 2: 791-797, 1996.

Chen, X., Lowe, M., and Keyomarsi, K. UCN-01-mediated G1 arrest in normal but not tumor breast cells is pRb-dependent and p53-independent. Oncogene, 18: 5691-5702, 1999.

Chou, T-C., and Talalay, P. Quantitative analysis of dose-effect relationships: the combined effects of multiple drugs or enzyme inhibitors. Adv. Enzyme Regul., 22: 27-55, 1984.

Cross, T. G., Scheel-Toellner, D., Henriquez, N. V., Deacon, E., Salmon, M., and Lord, J. M. Serine/threonine protein kinases and apoptosis. Exp. Cell Res., 256: 34-41, 2000.

Dai Y, Yu C, Singh V, Tang L, McKinstry R, Dent P, and Grant S, Pharmacologic inhibitors of the MEK/MAPK cascade interact synergistically with UCN-01 to induce mitochondrial dysfunction and apoptosis in human leukemia cells. Cancer Res 61: 5106-5115, 2001.

Davis, S., Vanhoutte, P., Pages, C., Caboche, J., and Laroche, S. The MAPK/ERK cascade targets both Elk-1 and cAMP response element-binding protein to control long-term potentiation-dependent gene expression in the dentate gyrus in vivo. J. Neurosci., 0: 4563-4572, 2000.

Dudley, D. T., Pang, L., Decker, S. J., Bridges, A. J., and Saltiel, A. R. A synthetic inhibitor of the mitogen-activated protein kinase cascade. Proc. Natl. Acad. Sci. USA, 92: 7686-7689, 1995.

Favata, M. F., Horiuchi, K. Y., Manos, E. J., Daulerio, A. J., Stradley, D. A., Feeser, W. S., Van Dyk, D. E., Pitts, W. J., Earl, R. A., Hobbs, F., Copeland, R. A., Magolda, R. L., Scherle, P. A., and Trzaslcos, J. M. Identification of a novel inhibitor of mitogen-activated protein kinase kinase. J. Biol. Chem., 273: 18623-18632, 1998.

Freemerman, A. J., Turner, A. J., Birrer, M. J., Szabo, E., Valerie, K., and Grant, S. Role of c-jun in human myeloid leukemia cell apoptosis induced by pharmacological inhibitors of protein cinase C. Mol. Pharmacol., 49: 788-795, 1996.

Fuse, E., Tanii, H., Takai, K., Asanome, K., Kurata, N., Kobayashi, H., Kuwabara, T., Kobayashi, S., and Sugiyama, Y. Altered pharmacokinetics of a novel anticancer drug, UCN-01, caused by specific high affinity binding to al-acid glycoprotein in humans. Cancer Res., 59: 1054-1060, 1999.

Gao Q, Singh L, Kumar A, Srinivasan S, Wazer D E, and Band V, Human papillomavirus type 16 E6-induced degradation of E6TP1 correlates with its ability to immortalize human mammary epithelial cells. J Virol 75: 4459-4466, 2001.

Gorczyca, W., Gong, J., and Darzynldewicz, Z. Detection of DNA strand breaks in individual apoptotic cells by the in situ terminal deoxynucleotidyl transferase and nick translation assays. Cancer Res., 53: 1945-1951, 1993.

Graves, P. R., Yu, L., Schwarz, J. K., Gales, J., Sausville, E. A., O'Connor, P. M., and Piwnica-Worms, H. The Chil protein kinase and the Cdc25C regulatory pathways are targets of the anticancer agent UCN-01. J. Biol. Chem., 275: 5600-5605, 2000.

Harvey, S., Decker, R., Dai, Y., Schaefer, G., Tang, L., Kramer, L., Dent, P., and Grant, S. Interactions between 2-fluoroadenine 9-b-D-arabinofuranoside and the kinase inhibitor UCN-01 in human leukemia and lymphoma cells. Clin. Cancer Res., 7: 320-330, 2001.

Jarvis, W. D., Povirk, L. F., Tirner, A. J., Traylor, R. S., Gewirtz, D. A., Pettit, G. R., and Grant, S. Effects of bryostatin 1 and other pharmacological activators of protein kinase C on 1-[b-D-arabinofuranosyl]cytosine-induced apoptosis in HL-60 human promyelocytic leukemia cells. Biochem. Pharmacol., 47: 839-852, 1994.

Jarvis, W. D., Fomari, F. A., Jr., Tombes, R. M., Erukulla, R K., Bittman, R., Schwartz, G. K., Dent, P., and Grant, S. Evidence for involvement of mitogen-activated protein idnase, rather than stress-activated protein linase, in potentiation of 1-b-D-arabinofuranosylcytosine-induced apoptosis by interruption of protein kinase C signaling. Mol. Pharmacol., 54: 844-856, 1998.

Kurata, N., Kuwabara, T., Tanii, H., Fuse, E., Akiyama, T., Alcinaga, S., Kobayashi, H., Ymaguchi, K., and Kobayashi, S. Pharmacokinetics and pharmacodynamics of a novel protein kinase inhibitor UCN-01. Cancer Chemother. Pharmacol., 44: 12-18, 1999.

Lebedeva I, Rando R, Ojwang J, Cossum P, and Stein C A, Bcl-xL in prostate cancer cells: effects of overexpression and down-regulation on chemosensitivity. Cancer Res 60: 6052-6060, 2000.

Leppa, S., and Bohmann, D. Diverse functions of JNK signaling and c-Jun in stress response and apoptosis. Oncogene, 18: 6158-6162, 1999.

Luo D, Cheng S C, Xie H, and Xie Y, Effects of Bcl-2 and Bcl-XL protein levels on chemoresistance of hepatoblastoma HepG2 cell line. Biochem. Cell Biol 78: 119-126, 2000.

Mizuno, K., Noda, K., Ueda, Y., Hanald, H., Saido, T. C., Ikuta, T., Kuroki, T., Tamaoki, T., Hirai, S., and Osada, S. UCN-01, an anti-tumor drug, is a selective inhibitor of the conventional PKC subfamily. FEBS Lett., 359: 259-261, 1995.

Park J S, Reardon D B, Carter S, Schmidt-Ullrich R K, Fisher P B, and Dent P, Mitogen Activated Protein (MAP) kinase pathway signaling is required for release/progression of cells through G2/M after exposure to ionizing radiation. Mol Biol Cell 10: 4215-4231, 1999.

Peng, C-Y., Graves, P. R., Thoma, R. S., Wu, Z., Shaw, A. S., and Piwnica-Worms, H. Mitotic and G2 checkpoint control: regulation of 14-3-3 protein binding by phosphorylation of Cdc25C on serine-216. Science (Wash. D.C.), 277: 1501-1505, 1997.

Peng, C. Y., Graves, P. R., Ogg, S., Thoma, R. S., Byrnes, M. J., Wu, Z., Stephenson, M. T., and Piwnica-Worms, H. C-TAK1 protein kinase phosphorylates human Cdc25C on serine 216 and promotes 1-3-3 binding. Cell Growth Differ., 9: 197-208, 1998.

Sebolt-Leopold, J. S., Dudley, D. T., Herrera, R., Van Becelaere, K., Wiland, A., Gowan, R C., Tecle, H., Barrett, S. D., Bridges, A., Przybranowski, S., Leopold, W. R., and Saltiel, A. R. Blockade of the MAP Idnase pathway suppresses growth of colon tumors in vivo. Nat. Med., 5: 810-816, 1999.

Segar, R., and Krebs, E. G. The MAPK signaling cascade. FASEB J., 9: 726-735, 1995.

Shao, R-G., Cao, C-X., Shimizu, T., O'Connor, P. M., Kohn; K. W., and Poumier, Y. Abrogation of an S-phase checkpoint and potentiation of camptothecin cytotox-icity by 7-hydroxystaurosporine (UCN-01) in human cancer cell lines, possibly influenced by p53 function. Cancer Res., 57: 4029-4035, 1997.

Shi, Z., Azuma, A., Sampath, D., Li, Y. X., Huang, P., and Plunkett, W. S-Phase arrest by nucleoside analogues and abrogation of survival without cell cycle progres-sion by 7-hydroxystaurosporine. Cancer Res., 61:1065-1072, 2001.

St. Croix, B., Florenes, V. A., Ralc, J. W., Flanagan, M., Bhattacharya, N., Slingerland, J. M., and Kerbel, R. S. Impact of the cyclin-dependent kinase inhibitor p27Kip1 on resistance of tumor cells to anticancer agents. Nat. Med., 2: 1204-1210, 1996.

Tang, L., Boise, L. H., Dent, P., and Grant, S. Potentiation of 1-b-D-arabinofaranosylcytosine-mediated mitochondrial damage and apoptosis in human leukemia cells (U937) overexpressing bcl-2 by the idnase inhibitor 7-hydroxystaurosporine (UCN-01). Biochem. Pharmacol., 60: 1445-1456, 2000.

Tibbles, L. A., and Woodgett, J. R The stress-activated protein kinase pathways. Cell. Mol. Life Sci., 55: 1230-1254, 1999.

Valerie K, Hawkins W, Farnsworth J, Schmidt-Ullrich R, Lin P S, Amir C, and Feden J, Substantially improved in vivo radiosensitization of rat glioma with mutant HSV-TK and acyclovir. Cancer Gene Ther 8: 3-8, 2001.

Verheij, M., Bose, R., Lin, X. H., Yao, B., Jarvis, W. D., Grant, S., Birrer, M. J., Szabo, E., Zon, L. I., Kyriakis, J. M., Haimovitz-Friedman, A., Fuks, Z., and Kolesnick, R. Requirement for ceramide-initiated SAPK/JNK signaling in stress-induced apoptosis. Nature (Lond.), 380: 75-79,1996.

Vrana, J. A., and Grant, S. Synergistic induction of apoptosis in human leukemia cells (U937) exposed to bryostatin 1 and the proteasome inhibitor lactacystin involves dysregulation of the PKC/MAPK cascade. Blood, 97: 2107-2114, 2001.

Wang, Z., Su, Z. Z., Fisher, P. B., Wang, S., VanTuyle, G., and Grant, S. Evidence of a functional role for the cyclin-dependent kinase inhibitor P21 (WAF1/CIP1/MDA6) in the reciprocal regulation of PKC activator-induced apoptosis and differentiation in human myelomonocytic leukemia cells. Exp. Cell Res., 244: 105-116, 1998.

Wang, Q., Worland, P. J., Clark, J. L., Carlson, B. A., and Sausville, E. A. Apoptosis in 7-hydroxystaurosporine-treated T lymphoblasts correlates with activation of cyclin-dependent kinases 1 and 2. Cell Growth Differ., 6: 927-936, 1995.

Wang, Z., Van Tuyle, G., Conrad, D., Fisher, P. B., Dent, P., and Grant, S. Dysregulation of the cyclin-dependent kinase inhibitor p21WAF1/CIP1/MDA6 in-creases the susceptibility of human leukemia cells (U937) to 1-b-D-arabinofurano-sylcytosine-mediated mitochondrial dysfunction and apoptosis. Cancer Res., 59: 1259-1267, 1999.

Wang S, Guo C Y, Dent P, and Grant S, Effect of Bcl-XL expression on taxol-induced apoptosis and cytotoxicity in human leukemia cells (U937). Leukemia 13: 1564-1573, 1999.

Wang, S., Vrana, J. A., Bartimole, T. M., Freemerman, A. J., Jarvis, W. D., Kramer, L. B., Krystal, G., Dent, P., and Grant, S. Agents that down-regulate or inhibit protein kinase C circumvent resistance to 1-b-D-arabinofuranosylcytosine-induced apoptosis in human leukemia cells that overexpress Bcl-2. Mol. Pharmacol., 52: 1000-1009, 1997.

Wilson, W. H., Sorbara, L., Figg, W. D., Mont, E. K., Sausville, E., Warren, K. E., Balis, F. M., Bauer, K., Raffeld, M., Senderowicz, A. M., and Monks, A. Modulation of clinical drug resistance in a B-cell lymphoma patient by the protein kinase inhibitor 7-hydroxystaurosporine: presentation of a novel therapeutic paradigm. Clin. Cancer Res., 6: 415-421, 2000.

Wobst et al. (1998) Ann. Oncol. 9, 951-962.

Wouters B G, Denlko N C, Giaccia A J, and Brown J M, A p53 and apoptotic independent role for p21waf1 in tumour response to radiation therapy. Oncogene 18: 6540-6545, 1999.

Xia, Z., Dickens, M., Raingeaud, J., Davis, R., and Greenberg, M. E. Opposing effects of ERK and JNK-p38 MAP kinases on apoptosis. Science (Wash. D.C.), 270: 1326-1331, 1995.

We claim:

1. A method for promoting apoptosis and reducing clonogenic survival in cancer cells, comprising the step of
co-administering to said cancer cells a cell cycle checkpoint abrogation agent and an agent that inhibits a compensatory cytoprotective pathway, wherein said cell cycle checkpoint abrogation agent and said agent that inhibits a compensatory cytoprotective pathway are present in a quantity sufficient to promote apoptosis and reduced clonogenic survival of said cancer cells.

2. The method of claim 1 further comprising the step of exposing said cancer cells to radiation.

3. The method of claim 1 wherein said cell cycle checkpoint abrogation agent is selected from the group consisting of UCN-01 and caffeine.

4. The method of claim 1 wherein said agent that inhibits a compensatory cytoprotective pathway is selected from the group consisting of an agent that inhibits MEK 1/2 pathway and an agent that inhibits PI 3 kinase pathway.

5. The method of claim 4 wherein said agent that inhibits MEK 1/2 pathway is selected from the group consisting of PD98059, U0126, PD184352 and SL327.

6. The method of claim 4 wherein said agent that inhibits PI 3 kinase pathway is selected from the group consisting of LY294002 and wortmanin.

7. The method of claim 1 wherein said cancer cells are selected from the group consisting of leukemia cells, prostate cancer cells, breast cancer cells, brain cancer cells, heptomas, colon cancer cells, myeloma cells, and lymphoma cells.

8. A method for treating cancer in a patient in need thereof, comprising the step of
co-administering to said patient a cell cycle checkpoint abrogation agent and an agent that inhibits a compensatory cytoprotective pathway, wherein said cycle checkpoint abrogation agent and said agent that inhibits a compensatory cytoprotective pathway are present in a quantity sufficient to ameliorate symptoms of said cancer in said patient.

9. The method of claim 8 further comprising the step of administering radiation to said patient.

10. The method of claim 8 wherein said cell cycle checkpoint abrogation agent is selected from the group consisting of UCN-01, and caffeine.

11. The method of claim 8 wherein said agent that inhibits a compensatory cytoprotective pathway is selected from the group consisting of an agent that inhibits MEK 1/2 pathway and an agent that inhibits PI 3 kinase pathway.

12. The method of claim 11 wherein said agent that inhibits MEK 1/2 pathway is selected from the group consisting of PD98059, U0126, PD184352 and SL327.

13. The method of claim 11 wherein said agent that inhibits PI 3 Idnase pathway is selected from the group consisting of LY294002 and wortmanin.

14. The method of claim 8 wherein said cancer is selected from the group consisting of leukemia, prostate cancer, breast cancer, hepatomas, brain cancer, colon cancer, myeloma, and lymphoma.

15. A method of radiosensitizing cancer cells, comprising the step of co-administering to said cancer cells a cell cycle checkpoint abrogation agent and an agent that inhibits a compensatory cytoprotective pathway, wherein said cell cycle checkpoint abrogation agent and said agent that inhibits a compensatory cytoprotective pathway are present in a quantity sufficient to radiosensitize said cancer cells.

16. The method of claim 15 wherein said cell cycle checkpoint abrogation agent is selected from the group consisting of UCN-01 and caffeine.

17. The method of claim 15 wherein said agent that inhibits a compensatory cytoprotective pathway is selected from the group consisting of an agent that inhibits MEK 1/2 pathway and an agent that inhibits PI 3 kinase pathway.

18. The method of claim 17 wherein said agent that inhibits MEK 1/2 pathway is selected from the group consisting of PD98059, U0126, PD184352 and SL327.

19. The method of claim 17 wherein said agent that inhibits PI 3 kinase pathway is selected from the group consisting of LY294002 and wortmanin.

20. A composition comprising,
a cell cycle checkpoint abrogation agent,
an agent that inhibits a compensatory cytoprotective pathway, and
a carrier suitable for in vivo administration and exposure to ionizing radiation.

21. The composition of claim 20 wherein said cell cycle checkpoint abrogation agent is selected from the group consisting of UCN-01 and caffeine.

22. The composition of claim 20 wherein said agent that inhibits a compensatory cytoprotective pathway is selected from the group consisting of an agent that inhibits MEK 1/2 pathway and an agent that inhibits PI 3 kinase pathway.

23. The composition of claim 22 wherein said agent that inhibits MEK 1/2 pathway is selected from the group consisting of PD98059, U0126, PD184352 and SL327.

24. The composition of claim 22 wherein said agent that inhibits PI 3 kinase pathway is selected from the group consisting of LY294002 and wortmanin.
